SEQUENCE LISTING (37 sequences)

SEQ ID NO: 1 - DNA, 347 bp, Sus scrofa
ggactccacctctgccttcctccagccacccctacccccaccacaagctgttgacagtctggccattcactccctgctcacattyccactcccggttctaaaaggggaaaacttgtcaaggacagtcttgacaaatgggtcacaggccacccttctatcactagtaaggagataggcagtcaggttggaacagaagaggttttgagaagcctgctggcttgcccaggctcacagcaggcaccggcctccaaggtcacatcccagaaggagataggggctggcctcccacacccacattcctgctcccccatataagccagggcagcccagcccctcaaagtgccagg

SEQ ID NO: 2 - DNA, 459 bp, Sus scrofa
atgcagggtccttctctctctcagctggtcctggtgctggcagcaatgggggccctgctggaggctgggacccccagagaagaggtctccagcaccccagctttgcccagggagccagccacaggcaccgaggggctcatcttccactgggactggaactggccgccccctggtgcctggcccctgggtggccctcaggaccccctgtgcctagtgaccctgaatggagaccctggcaatgggagcagtccttttctgtgggtggtggggactctaagcagttatgagcaggccttcctggaggctgtgcggcatgcccgctggggtccccaagacctggccaactttgggctctgccctcccagcctcaggcaggctgccctgccccttttgcagcagctgcaggcatggctgggggagcccagggggcagcgactggtggtcctgcacctggaggaa

SEQ ID NO: 3 - DNA, 610 bp, Sus scrofa
tgccctgccccttttgcagcagctgcaggcatggctgggggagcccagggggcagcgactggtggtcctgcacctggaggaaggtacgtgggggctgcagcgggacctggtgggtgggcagaggactgggctctagtctcaggatgggagacgactgtttcttgcytagagccgcacccagcctcctcaggaagttgaggctgatggccagacaggtgggtgaccttattttgccctgtctgggagtgcctcctccagtacctgggaaggtccagcaacagacaaatacacaygrccatggacctcagggacccactgcagggaakggcttccctccaggagagcttcagaccaagagaccccaagggcttgggtaacccacagcagtgggggcagtgctccaccacccaccctatgcatccctcctcccaggttgcctgtcccaggcaggtttggcacctggagcccaagggtatcaagtgtctctcagacacagagccgtccccccactgaggctccccctcctgcacaggcgacaggctttgggggagggtcttgggcttctgtggttcaggcaactctgtccacttccccctttgtcctggccaca

SEQ ID NO: 4 - DNA, 144 bp, Sus scrofa
gtgtcgtgggagccaacacccttgctgaagttccaggagcccctacctggagaagccagccccctggagctggcgctgttggtgttgtatccagggcccggcccagaggtcactgtcaccggggctgggctgcccggcgcccag

SEQ ID NO: 5 - DNA, 212 bp, Sus scrofa
gtaccagagaggtgaatgaggctgtccctgggccaccaggagccctcattcaaggcaagggcgggattattgagggggggggggktaactgcacctaacagaaaggctgtgactgtccaagttggaattttgcagggatgtttagggcagcaggcaagcagggctggtgtcccaaggccccagcaagcctggctgagtccccatctccacag

SEQ ID NO: 6 - DNA, 108 bp, Sus scrofa
agcctctgcccgacyagggactctggcttcctggcgttggcggtcgaccacccagagagggcctggcgtggctctgggcttgctctgaccctgcgacgccgcggaaat

SEQ ID NO: 7 - DNA, 105 bp, Sus scrofa
ggtagccccctcccccagactggagccgggctggggcggctgccctcggaaacaccccccccaccacccttccagycgstgagccagctcctgcctccatcctca

SEQ ID NO: 8 - DNA, 162 bp, Sus scrofa
ggtgcctccctgagcaccgcccagctgcaggcgctgctgtttggcgccgactctcgctgcttcacacggatgaccccggccctgctcctgttgccgccgcaggggccggtgccgatgcccgcacacggccgggtggactcaatgccattcccgcagcccagg

SEQ ID NO: 9 - DNA, 101 bp, Sus scrofa
ctgcgcatgagtcagaacttgggggcgcagggacgtgggggcagcgcaggcttgtgccctcacgtccccgcgctccgccgtccaggctgtccccagagccc

SEQ ID NO: 10 - DNA, 22 bp, Sus scrofa
ggactccacctctgccttcctc

SEQ ID NO: 11 - DNA, 22 bp, Sus scrofa
ggaacttcagcaagggtgttgg

SEQ ID NO: 12 - DNA, 21 bp, Sus scrofa
ccagcaacagacaaatacacg

SEQ ID NO: 13 - DNA, 20 bp, Sus scrofa
gctccaggtgccaaacctgc

SEQ ID NO: 14 - DNA, 22 bp, Sus scrofa
ggatgtttagggcagcaggcaa

SEQ ID NO: 15 - DNA, 19 bp, Sus scrofa
gcggcgtcgcagggtcaga

SEQ ID NO: 16 - DNA, 18 bp, Sus scrofa
ctgcgacgccgcggaaat

SEQ ID NO: 17 - DNA, 22 bp, Sus scrofa
gatggaggcaggagctggctca

SEQ ID NO: 18 - DNA, 152 bp, Sus scrofa
gagccagggagtgatgctgagatcaaagaatttgctgctggctacaacgtcaaatttgatatgttcagcaagatctgtgtgaatggggacgatgcccaccctctgtggaagtggatgaaagtccagcccaaggggaggggcatgctgggaaa

SEQ ID NO: 19 - DNA, 86 bp, Sus scrofa
gtgagttggggggctggggtgagagtggagggcagtggggatctgcagctgccacgggattactgatracacatttctttttgcag

SEQ ID NO: 20 - DNA, 25 bp, Sus scrofa
tgctatcaaatggaactttaccaag

SEQ ID NO: 21 - DNA, 160 bp, Sus scrofa
gtaagggggtgctgagggccyggggggtgccctcagtcaccctggtgccacttctagggtctccacctgacctaaatggagtgatgggtgggggccgcttgcttgcttgccccagtcccaccacggtggccttctgtccctgacaccacctgtcctgcag

SEQ ID NO: 22 - DNA, 60 bp, Sus scrofa
ttcctcattgataagaacggctgtgtggtgaagcggtacggtcccatggaagagccccag

SEQ ID NO: 23 - DNA, 22 bp, Sus scrofa
cagctgccacgggattactgtt

SEQ ID NO: 24 - DNA, 20 bp, Sus scrofa
cccccacccatcactccatt

SEQ ID NO: 25 - DNA, 873 bp, Sus scrofa
atgcagggtccttctctctctcagctggtcctggtgctggcagcaatgggggccctgctggaggctgggacccccagagaagaggtctccagcaccccagctttgcccagggagccagccacaggcaccgaggggctcatcttccactgggactggaactggccgccccctggtgcctggcccctgggtggccctcaggaccccctgtgcctagtgaccctgaatggagaccctggcaatgggagcagtccttttctgtgggtggtggggactctaagcagttatgagcaggccttcctggaggctgtgcggcatgcccgctggggtccccaagacctggccaactttgggctctgccctcccagcctcaggcaggctgccctgccccttttgcagcagctgcaggcatggctgggggagcccagggggcagcgactggtggtcctgcacctggaggaagtgtcgtgggagccaacacccttgctgaagttccaggagcccctacctggagaagccagccccctggagctggcgctgttggtgttgtatccagggcccggcccagaggtcactgtcaccggggctgggctgcccggcgcccagagcctctgcccgaccagggactctggcttcctggcgttggcggtcgaccacccagagagggcctggcgtggctctgggcttgctctgaccctgcgacgccgcggaaatggtgcctccctgagcaccgcccagctgcaggcgctgctgtttggcgccgactctcgctgcttcacacggatgaccccggccctgctcctgttgccgccgcaggggccggtgccgatgcccgcacacggccgggtggactcaatgccattcccgcagcccagg

SEQ ID NO: 26 - DNA, 2241 bp, Sus scrofa
  misc_feature (85)..(85): n is a, c, g, or t
ggactccacctctgccttcctccagccacccctacccccaccacaagctgttgacagtctggccattcactccctgctcacattnccactcccggttctaaaaggggaaaacttgtcaaggacagtcttgacaaatgggtcacaggccacccttctatcactagtaaggagataggcagtcaggttggaacagaagaggttttgagaagcctgctggcttgcccaggctcacagcaggcaccggcctccaaggtcacatcccagaaggagataggggctggcctcccacacccacattcctgctcccccatataagccagggcagcccagcccctcaaagtgccaggatgcagggtccttctctctctcagctggtcctggtgctggcagcaatgggggccctgctggaggctgggacccccagagaagaggtctccagcaccccagctttgcccagggagccagccacaggcaccgaggggctcatcttccactgggactggaactggccgccccctggtgcctggcccctgggtggccctcaggaccccctgtgcctagtgaccctgaatggagaccctggcaatgggagcagtccttttctgtgggtggtggggactctaagcagttatgagcaggccttcctggaggctgtgcggcatgcccgctggggtccccaagacctggccaactttgggctctgccctcccagcctcaggcaggctgccctgccccttttgcagcagctgcaggcatggctgggggagcccagggggcagcgactggtggtcctgcacctggaggaatgccctgccccttttgcagcagctgcaggcatggctgggggagcccagggggcagcgactggtggtcctgcacctggaggaaggtacgtgggggctgcagcgggacctggtgggtgggcagaggactgggctctagtctcaggatgggagacgactgtttcttgcntagagccgcacccagcctcctcaggaagttgaggctgatggccagacaggtgggtgaccttattttgccctgtctgggagtgcctcctccagtacctgggaaggtccagcaacagacaaatacacacgaccatggacctcagggacccactgcagggaanggcttccctccaggagagcttcagaccaagagaccccaagggcttgggtaacccacagcagtgggggcagtgctccaccacccaccctatgcatccctcctcccaggttgcctgtcccaggcaggtttggcacctggagcccaagggtatcaagtgtctctcagacacagagccgtccccccactgaggctccccctcctgcacaggcgacaggctttgggggagggtcttgggcttctgtggttcaggcaactctgtccacttccccctttgtcctggccacagtgtcgtgggagccaacacccttgctgaagttccaggagcccctacctggagaagccagccccctggagctggcgctgttggtgttgtatccagggcccggcccagaggtcactgtcaccggggctgggctgcccggcgcccaggtaccagagaggtgaatgaggctgtccctgggccaccaggagccctcattcaaggcaagggcgggattattgagggggggggntaactgcacctaacagaaaggctgtgactgtccaagttggaattttgcagggatgtttagggcagcaggcaagcagggctggtgtcccaaggccccagcaagcctggctgagtccccatctccacagagcctctgcccgaccagggactctggcttcctggcgttggcggtcgaccacccagagagggcctggcgtggctctgggcttgctctgaccctgcgacgccgcggaaatggtagccccctcccccagactggagccgggctggggcggctgccctcggaaacaccccccccccttccagncgntgagccagctcctgcctccatcctcaggtgcctccctgagcaccgcccagctgcaggcgctgctgtttggcgccgactctcgctgcttcacacggatgaccccggccctgctcctgttgccgccgcaggggccggtgccgatgcccgcacacggccgggtggactcaatgccattcccgcagcccaggctgcgcatgagtcagaacttgggggcgcagggacgtgggggcagcgcaggcttgtgccctcacgtccccgcgctccgccgtccaggctgtccccagagccc

SEQ ID NO: 27 - DNA, 483 bp, Sus scrofa
gagccagggagtgatgctgagatcaaagaatttgctgctggctacaacgtcaaatttgatatgttcagcaagatctgtgtgaatggggacgatgcccaccctctgtggaagtggatgaaagtccagcccaaggggaggggcatgctgggaaagtgagttggggggctggggtgagagtggagggcagtggggatctgcagctgccacgggattactgatgacacatttctttttgcagtgctatcaaatggaactttaccaaggtaagggggtgctgagggcccggggggtgccctcagtcaccctggtgccacttctagggtctccacctgacctaaatggagtgatgggtgggggccgcttgcttgcttgccccagtcccaccacggtggccttctgtccctgacaccacctgtcctgcagttcctcattgataagaacggctgtgtggtgaagcggtacggtcccatggaagagccccag

SEQ ID NO: 28 - DNA, 5662 bp, Sus scrofa
gaattcaggaaagaggtcttctgttcatttaaaatataacgtgatgtgtgttaacactgaggtagatactgggaattaaggaaacaatagaaagtactggactgagaatgaatacggaatactgtgtaaagtggaacgagtgaatgtctcctaggggaagctacatctaaatggaatcttgtagaagtgtttgtaggaatagctcagatgaaaaggagatgaaaaaggtacctcaggcttaaggaatagcctgattttcagaggtgggaaggtgcttcaagccaatgaagtgagattttttttttttttggtctttttagggttgcacccacagcatatggaagtttccaagctaaggtcgaattggaactgcaactgccaacctacgccacagtcacagcaacatgggatctgagctgcatctgtgaactacactgcagctcatggcaacaccagatccttaacccactgagcaaatctagagatcaaacctgtgccctaatggatactagccaggttcactaccactgagccacaacggtaactactgacgtgagaatttaacataggacctccttaaataatgttcaacattttgtttaaatattgagttaattaatattattatactagaacccagtaataaagggctagaaataaaaatgggtattatcagtcaccttctaaccaggaaaacagaaactgctcctgataagagaagtcagaggatatttaatctggggaatgcattacctaagttttagaattgttgagaagccagacaggaaataaggaaacccaaaaatcagtaaccatgggaagctcccatctaccctcaggattagagagacacaaatgaggttcctggagccaaaaggtgagaccacccagcagaagctcaagccacatgtggagtttcctcacaaaagctgggaacactgagggaggagctgtctgatgcaacctggaccaagggagaaagtgcagctactgacaaggaaagaatgtaaaggagagacatactccaaccttcttcttcttttcactctctaatctccttccacagagacaaaaggctgctgacacagcagcctaagaaaggtagcctgcagaggtcccttctcccaaaaatcagagagcaaaacaggacaagaacaaaaaatgtatcagatagcaaacaggctatggacaagcacaacagaaagaaaatcagagtgatctatgtttcacttagttcaacaaaagtgtatcagtgctggagttccccttgtggctcagcaaaaacaaacctgactagtatccatgaggactcacattccatccctggcctcactcagtgggttaaggatccagcattgccatgagctatggtgtaggctgcagactcagctcagatctggtattgctgtggctatggtgtaggccggacggtacagctccgattcgaccccacctgagaatttccatatgccacaagtgcggccctaaaaagacaaaaaaaaaaaaaaaaaaaaaaaaaaaaaagagtattagtgcctactgtggattagaaaccattcctattgggaatacaaaggtgaataagaaagctcactacatcttcatcaataaaatattttaataacttttgtgagagcagtaacatctaacttggaaatacacttatcagataaactagactataagaggcttgacattgtgagaaaggtctggggcctcgtgataggtcaaggaaaataaggttatttggggaaacctcagacctaaatgtggatggaagtataaatatggacattaggaatagcttcccaaattctggatggcctctgttttggcccctctccaactaatgcagttggtgagaattataaaccacagtatggttcaatgagtagctctgttttggagaccagcagacctagatatgaaccttagccttgctctttcaggttccatagttttgggcaagtcatttaaatgttttcccatctttcaaagagtaataatagtaactcctttaaaaagttgtttaaaaattatatgtgatcatatatttgaagtgtttaagtgtctggggcatagtaggtgctcaataaaaacctgttaatattttaaattgaatgtgaaaagattgtatatacattactcattaaaacacatgaattcaatatagtcatataaatatactttgtgaacacgcatagataacataaaaagagttaatttgaaatataaggtgggaattcgtaccatggcacagggggttaatgatccacttgtctctgtggcattgctggctcaattcccagcctggctcagtgggtaggatctggcattgccacagctatggcataggccacagattcaactcggattcgatccctagcctggaaacttccacatgccacaggtgcagccattaaaaaaaaaaaaaaaaaattctacattccttattacttacacaagtgctaaatcagcccccagtactttgataagttttatctttgtcacacatgtttgataaaatcataaccctggataaatccaagtatttgttacccatgagtctgaactcctgccattaaattaggcaaaaaaaaaaaaaaaaaaaaaatcatgtttagtggtcttgggttaaatttttttaccataaacctcaaatggtcccttaatactggtaggcaattttactacctatacctaactcaccaatgactcagtccctctaccagtctcatacaaatattaagccttggatctctcaatcctcaacaatgcatccactacctttactctcagatgatgatcttacttcctactttactgagaaaatgaaaacaatgacaggagagtctgtataaagcccatcacccaccaaacactcaccatcttctgcactcaccacacctccccaaccagcagcatctctacccatgactctgcctcctgcccacaacaggatgagctctcctgtttaaagccagtcattctacttgtgctctaagatccatctcttctcatagtctacctaagaacactgaagaaattttcctctcttgctccaacatcatttttctctcaatcatttgcatcaccaaactaacagttatgtcttcagtcttaaaacataaaaatcaaaaggaaattatctttaccccacttccatgtgaccaaatcacctgtttttttcctcatctttgtatcaaaattctggggagaaaaagttcaacactttttttgtaatggtcacacctgtggcatataggagctcctttgccacagccacagtaatgccggacccaagttgcatctgcaactgacacgcagtttatggcaatgcggatccttagtccactgagagaggccagggattgaatttatatcctcaggaaaacaatgctgggttcttaacttgctgagccaaatgtgaactcctcaattcttttttattcatttctttccaatcactcagtctgctcttttattgaattatagctgatctataatggtatgttagtttctggtgtatagcaaagtgattcagttatacatacatattacttttcacattcttttccatgacagtttatcacaggatattgaatatagttgcgatacagtaggaccattttgtttatctatcctatatataatagtggttaatcccaaagtcccaatccaaaccatccccaccctcctgcccttggcaactacaagtctgttctccatgtctgtgagtctgtttctgttccattcatttgtgtcataatttagattccacatataattgtaatcatatggtatttgtctttctctttctgacttgcctcacttagtatgacaatccatgtagccacaaatgtcttgacaattacttaaacacacaccaatcagggttttgtttctctcactccaaaggagcttctctagccaaggacactggcaacatttatgctgccacacgcattgctaacctgtcagcagcatttggtacagttgtcacttgctcctcctgacaaactggctttacttgatttctgggacaccacattctctccattcctttctttcctcaatgacccttctgtttcctttgggcaaaggaagggaaaaaaacttcatcttattcttgacctcttaatattagcacacaccagcctccactcttggtccttttatcttctctatttatacttactcccttggtaacttcttcaaggctcatgccaattatacattttagctagcatatttctcccaaaatccagattcaccattctacttagatatcttaagctcaacctatccataccgaactccttatcattttcccaaacttactatatttatagccatcccatttcagttgataacaaattcatccttcaagtcactcaggccagaatctttagagtcatcttcactcttttctttttctcacactcaggattcatccatcagaaaatcctgctggctccactttcaaaatacatatgaaatcagattactttgattattttattactactattactgaacagatagcacttctcacccaagttgctgcaagagcatctaataggacttcctgtttctacctcccccacccccatattagcaaccaggcagccagagggtcctttaagacttaaacctgattatatcactcctatactcaaaaccctgcaactggtccccaaacaccgacagtaaaaactgaagtctttacattgaactaaaaagtccgacattatttgacttctgccacatctgtgacatcatatcctcatatttccatcattgttccttttctccagccaaggagcttaattaattaataagcttaattaattgctcaattaataaatatttgttaaatcaatctcagtttccatggagctcatagtctactgggagagaaaaatatataaaagaatacaaaaagaaggtaattaaagctttcctcaatctcccattcctaaacaatgacaagtgaatgttgaaggttgagaaatttgccagggggtgggagtagtataggggacattgggaggaagcaaggacatttcaggaaggatgaacatggcacatacaaagacctagagaaatgaatcagcagaacatttaaagaattacgagtaagcatcaaagaataatatttaagattaaggaatctgaatatgggaagtaaacataaatataatttacactttataaaagagtataatcatgaaagactctctatttgtttcttcccttacagctgtcagtctagtctcagagtaacttattaaccatatatatatatattttttgacacacctcaacagtgccaaagcaatacttggaaaggattctaaattccccaaattaaatatacaaaagaaaaacccagagtcagacttaatttgaaaaggtaaaggagtgggtgttctactatatcaaatttaatttgtacaaaatcatctctggtaacattatttttcctgttccactgtgtttagactactttagtaaggcttgatctccctgtctatctaaacactgattcacttacagccagcttcaggctaacattgatcttactaatacccaacaaatccacaaagtgttagtttcacatgattttgtataaaaggtgaactgagactagattcagccc

SEQ ID NO: 29 - DNA, 33 bp, Sus scrofa
acagcttcccccagacaaggcagccgatcacag

SEQ ID NO: 30 - DNA, 1205 bp, Sus scrofa
gtgagtcttagcatttatagttaccaagaggtgacagttagttctgaaatgatttttcgggatctgaagaacaaatctagagctttttaacttctgttggggagggaattcgtacttgtcaacctggcttctcaaatatggatagtgcactgtaattactgtagcaagcaattgacttttcatagaccagttcacctagcctctgatatggtcttattttacaaaaaggaggaaaaagcaaatgatatttatgagatgctaaaaatgatgaactaatttagtagtacaaaagtttttcttggagttcccatcgtggcgcaatggttaacgaatccgactaggaaccaagaggttgcgggttcgatccctggccttgctcagtgggttaaggatccagcattgctgtgagctgtggtgtaggttacagacacagcttggatcccacgttgctgtggccctggcatagggcgatggctacagctctgattagacccctagccttggaaactccatatgccaagggagcagtccaagaaatggcaaaaagaccaaaaaaaaaagtttttctttttaaataaaatgttttaaaatgataatgaagggacaaatatgatgatcacaattacttgcttcagagtaatcctttaagacagtcaatggcaatactctataaatattgctctgcttaaaacattatattggagttttgacccataatatagttctactttgacaaaaaaaaaaaaaattgaggaggagaataagaagaaacgtttggagttccccgtcgtggcgcagtggttaaacgaatccgattaggaaccatgaggttgcgggttcggtccctgcccttgctcagtgggttaatgatccggcgttgcatgagctgtggtgtaggttgcagacgaggctcggatccccgcgttgctgtggtttctggcgtaggcgggtggctacagttttgattcgacccctagcctgggaacctccatatgccgcggggagcgcccaaagaaatggcaaaagacagaaaaaaaaaaaaaaaaaaaaaaaaaaaaaaagaaacgtttgttcaagaaacaagaattaagaaaaggaaaggaaggaaaaccactatggagtaaaagtgactggagaggatgaatagaccagttattcaaggtttggtcaacttacattacgaatgtaattctttggtttttcag

SEQ ID NO: 31 - DNA, 197 bp, Sus scrofa
ttttttacaggccttaattgtttggtttccaccccaagatgaagtcgctgcagttttgcttcctattctgttgctggaaagccatctgctgcaatagctgtgagctgaccaacatcaccatcacagtggagaaagaggagtgtaacttctgcataagcatcaacaccacgtggtgtgctggctattgctacacccgg

SEQ ID NO: 32 - DNA, 1556 bp, Sus scrofa
gtaggttctttgctttgctagaagtgagggtgctgaaggtctgtaaaaggcgggctttactaattcccactttatcaatattttaagtttccggaacagccatgagtcccttagtcaatactgtctgtttcctgattggggttatttaccatgacatcggttaaatcttcaggcctggatttgattaaggtaaatttagggaagcctcagattttatctgattaatttggtaattgccaactctattttttaattttatttaatttttttatttcaaaaaaagtagttctattctagattctacacatacagagataaacacataaacatacatatatttaataacagaagatctacaatatttcccaaaagccaatttttgtaattgaagctatatctttgcaatagagatagtatcaaaatgtttgtagcaacataaaaacacagccatgttataaaaactgtcttactggcccatctcaatacaaatgccaacgcgcagcctgagaacacaatcaatccttgcagactgttaggacccaaatgaactggcaaacccactcccttctttatatggttgagaaaaacaaggcacagagggataaaaccactagtttgtattcacacagtttctttgaattaatccaagtgaaaaagcagtttctactttattttttcccctataacacctggatatcgatgcagaatttccgtaaattgaaattgaaaacaactttttaatgcaatatactttactgggtggtaaatgagtttgaccaaactccacttattgcatcttattgggatacagacttgatggcatgatatggaaataaattaaacataagtgtctatttcttccctcagtggatttttttttttaactagaaagtgttagaataaggttgttctgacaggactgaagttcttatacacaaacatgaaagctttgaaactgagctctgaaaaatatacagcatttaagaggggaagatgtctgtaagacagcagaatatttaaaatcttacatgaatttttatagtcatgttaagctaagtattaacattccacattatatatttttgattttttttatacacacccagggaccatgtattgagaaaatttttctgagaaattaaacttcagttttttatgggttaagctgtcattaatatagctttcaacttagtaattaatatagctttcaactttcaaaacgtcaaaatttctgtcctattttctttttaattattttttatattgaaagttaagtttctttaaagtcagagaaataattaacattttgacatagacataaggagtaggaaaaggaataatacattttctgtaagatttccagatcagaaaacatggcatagcatataggttatttatgatttatgaaatcatgtttccttggttaggaattctataaatggccttaatggataaatgtcagagcaagaaatattcaatgcctgtctcattttgattaaatagaaacttctgtaatactttaacctaactctctctctctcccctgaatcccttag

SEQ ID NO: 33 - DNA, 812 bp, Sus scrofa
gacctggtatacaaggacccagccaggcccaacatccagaaaacatgtaccttcaaggagctggtgtacgagaccgtgaaagtacctggctgtgctcaccatgcagactccctgtatacgtatccagtagccactgaatgtcactgtggcaagtgtgacagtgacagtactgactgcaccgtgagaggcctggggcccagctactgctccttcagtgaaatgaaagaataaagagcagtggacatttcatgcttcctacccttgtctgaaggaccaagacgtccaagaagtttgtgtgtacatgtgcccaggctgcaaaccactatgagagaccccactgatccctgctgtcctgtggaggaggagctccaggaatgcagagtgctagggcctcagtcccatcaccactcaaccctgtattttgggtctggttccataagttttattcggtcttttttttttaaattactcaatgaattttattacatttataattgtacaatgatcatcacaacccaattttataggatttccatcccaaacccccagcatagacccccatctcccaatctgtctcatttggaaaccataagtttttcaaagtccgtgagtcagtatctactcagtcttattaccttaatgacatgtgggtgttttctgtttaataatcttagaaatcctctcaagacagggatatggacccagaggaaggaaatgggctaagaatgggtgaaaggactaaatgcagcattctcccactagacacagaagcctacaagagcagggccagtctctttgtcatgagtgtggcc

SEQ ID NO: 34 - DNA, 994 bp, Sus scrofa
tcaatacctagcacagtgactagaattcagtaagaaactcaagaatggcttccttaaggaaagtaagattggaaatgtagggggtaggaaaatactgaaagaagatgttggaggctatgtgatgaggctgcccttggcaatgccagtcagcccgtggaagggggtccatcagttccagtaccgcttcaccgctcttcctccggcatatggaggatggagacaggacatctctctcaggcaggtggcggttaccgagctcaggatttccaacccctttagttaagggcaaaagcaagaaatgttaatgcgggtttgtggaaattaacccacatctattccatcatttaaataaatggaacaaatgctatcagactcctgcaaaactccctccaggttgggatccactcctttggagagaggtggatttgaaagcaggtttaaaagcgattttggcaacttaataagtacatttatcttatctaaaaatgcatttgtgtaaagaaatagctcttttagaattagccataaggggaaaaaaacaaacaaaaaaaactgctgttttctagaatactctatcagtcttttgtctatccatgttctcacaaatctatttctttcaagaaggtaaatcttgaagctatttcatgagttgatgttgttttaagatgttacctcttagttatgtacttgtttcatacttatgttgtttaatttatttaaatcttatttttttaataaagacgctagctactagagtcatagatttggatttttttcatataccagcagatgactaaaatgtctgtatatttataatattaatagaaagagtcttatttaaaaaaactccttggagttcccgtcgtggcgcagtggttaacgaatccgactaggaaccatgaggttgcgggttcggtccctgcccttgctcagtgggttaacggtccggcgttgccatgagctgtggtgtaggttgcagacgcggctcggatcc

SEQ ID NO: 35 - DNA, 294 bp, Sus scrofa
ggagttccccgtcgtggcgcagtggttaaacgaatccgattaggaaccatgaggttgcgggttcggtccctgcccttgctcagtgggttaatgatccggcgttgcatgagctgtggtgtaggttgcagacgaggctcggatccccgcgttgctgtggtttctggcgtaggcgggtggctacagttttgattcgacccctagcctgggaacctccatatgccgcggggagcgcccaaagaaatggcaaaagacagaaaaaaaaaaaaaaaaaaaaaaaaaaaaaaagaaacgttt

SEQ ID NO: 36 - DNA, 22 bp, Sus scrofa
cctttaagacagtcaatggcaa

SEQ ID NO: 37 - DNA, 22 bp, Sus scrofa
agtggtttttccttccttttcc

(19) United States
(12) Patent Application Publication
(10) Pub. No.: US 2004/0126795 A1
(43) Pub. Date: Jul. 1, 2004

(54) Genetic markers associated with scrotal hernias in pigs

(76) Inventors: Tun-Ping Yu, Berkeley, CA (US); Nicholas G. Hopkins, Berkeley, CA (US); Shoji Sasaki, Franklin, KY (US); Lizhen Wang, Franklin, KY (US); Johannes W. M. Bastiaansen, Rosmalen (NL); Nader Deeb, Berkeley, CA (US); Eldon R. Wilson, Franklin, KY (US); Alan J. Mileham, Berkeley, CA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/679,064

(22) Filed: Oct. 3, 2003

Related U.S. Application Data
(60) Provisional application No. 60/416,211, filed on Oct. 3, 2002.

(51) Int. Cl. C12Q 1/68

(52) U.S. Cl. 435/6

(57) ABSTRACT

A method for screening for scrotal hernias in animals is disclosed. The method involves assays for genetic differences in the MIS, GPX4A, and FSHb genes of the animal which are associated with scrotal hernias.

### CROSS-REFERENCE TO RELATED APPLICATIONS

This application claims benefit under 35 U.S.C. § 119(e) of provisional application 60/416,211 filed Oct. 3, 2002.

### FIELD OF THE INVENTION

This invention relates generally to the detection of genetic differences among animals. More particularly, the invention relates to genetic markers in pigs which have been identified in several genes and which are indicative of heritable phenotypes associated with deleterious traits, namely scrotal hernias. Methods and compositions for use of these markers in genotyping of animals and selection are also disclosed.

### BACKGROUND OF THE INVENTION

Congenital abnormalities in economically important animals, such as pigs, can lead to reduced production, disease and even death. In pigs, the rate of congenital defects, of which more than one hundred types have been recorded (Huston et al., Veterinary Bulletin 48:645-675 (1978)), is thought to be between 1 and 5%. Congenital defects in pigs include pityriasis rosea, splayleg, atresia ani, crytorchidism, intersexuality, shoulder and back deformities, congenital tremors and hernias.

Hernia or rupture is the protrusion of the intestines, or any other organ, through a natural or artificial opening in the body wall. A hernia is classified according to the part of the body in which it is located. The kinds of hernias commonly found in swine are (i) inguinal, in which the inguinal canal serves as the hernia ring, (ii) umbilical or navel, in which the umbilical or navel opening is the hernial ring, and (iii) ventral, in which the hernial ring is located in the lower part of the abdomen. Warwick, Wisconsin Agricultural Experiment Station Bulletin 62:1-27 (1926).

Of these defects, scrotal hernia (SH), which is a type of inguinal hernia, where a section of intestine protrudes into the scrotum, is one of the most economically important and is thought to occur at a rate of about 1-2% of piglets born in US production systems. Economic losses result from the following: 1) increased mortality in newborn males at castration due to poor surgical repair of the hernia; 2) finishers refusal to pay for herniated pigs arriving from nursery units (normal value in 2002 of nursery pig=˜$35); 3) slaughter plants only pay approximately half the normal carcass value (about $50 in the US in 2002) for herniated pigs arriving from finishers as they assume that the pig has not been castrated and is thus prone to boar taint.

The exact cause of SH is not known, but there is agreement that development of this defect is genetically influenced (Vogt and Ellersieck, Am. J. Vet. Res. 51:1501-1503 (1990)), and it is assumed that a small number of genes impact the condition. One study suggests that the heritability of SH is around 0.3 in three breeds of pig (Vogt and Ellersieck, Am. J. Vet. Res. 51:1501-1503 (1990)). The same authors found significant differences between breeds and between sires within breeds for SH.

One previous study (Didion, WO 96/39538) claimed to have found an association between a microsatellite on pig chromosome 6 and SH.

Genetic differences exist among individual animals as well as among breeds which can be exploited by breeding techniques to achieve animals with desirable characteristics. For example, Chinese pig breeds are known for reaching puberty at an early age and for their large litter size, while American breeds are known for their greater growth rates and leanness. Often, however, heritability for desired traits is low, and standard breeding methods which select individuals based upon phenotypic variations do not take fully into account genetic variability or complex gene interactions which exist.

There is a continuing need for an approach that deals with selection against incidence of scrotal hernias at the cellular or DNA level. This method will provide the ability to genetically evaluate animals and to enable breeders to more accurately select those animals which not only phenotypically express desirable traits but those which express favorable underlying genetic criteria. This has largely been accomplished to date by marker-assisted selection.

RFLP analysis has been used by several groups to study pig DNA. Jung et al., Theor. Appl. Genet., 77:271-274 (1989), incorporated herein by reference, discloses the use of RFLP techniques to show genetic variability between two pig breeds. Polymorphism was demonstrated for swine leukocyte antigen (SLA) Class I genes in these breeds. Hoganson et al., Abstract for Annual Meeting of Midwestern Section of the American Society of Animal Science, Mar. 26-28, 1990, incorporated herein by reference, reports on the polymorphism of swine major histocompatibility complex (MHC) genes for Chinese pigs, also demonstrated by RFLP analysis. Jung et al. Animal Genetics, 26:79-91 (1989), incorporated herein by reference, reports on RFLP analysis of SLA Class I genes in certain boars. The authors state that the results suggest that there may be an association between swine SLA/MHC Class I genes and production and performance traits. They further state that the use of SLA Class I restriction fragments, as genetic markers, may have potential in the future for improving pig growth performance.

The ability to follow a specific favorable genetic allele involves a novel and lengthy process of the identification of a DNA molecular marker for a major effect gene. The marker may be linked to a single gene with a major effect or linked to a number of genes with additive effects. DNA markers have several advantages; segregation is easy to measure and is unambiguous, and DNA markers are co-dominant, i.e., heterozygous and homozygous animals can be distinctively identified. Once a marker system is established, selection decisions could be made very easily, since DNA markers can be assayed any time after a tissue or blood sample can be collected from the individual infant animal, or even an embryo.

The present invention provides genetic markers, based upon the discovery of polymorphisms in the porcine MIS and GPX4A genes, which correlate with scrotal hernias in pigs. This will permit genetic typing of pigs for their MIS and GPX4A alleles and for determination of the relationship of specific polymorphisms to incidence of scrotal hernias. Thus, the markers may be selection tools in breeding programs to develop lines and breeds that produce offspring without scrotal hernias. Also disclosed are novel porcine MIS and GPX4A genomic sequences, as well as primers for assays to identify the presence or absence of marker alleles.

According to the invention, polymorphisms were identified in the MIS and GPX4A genes which are associated with the incidence of scrotal hernias in pigs.

It is an object of the invention to provide a method of screening pigs for scrotal hernias.

Another object of the invention is to provide a method for identifying genetic markers associated with scrotal hernias.

A further object of the invention is to provide genetic markers for selection and breeding to obtain pigs without scrotal hernias.

Yet another object of the invention is to provide a kit for evaluating a sample of pig DNA for specific genetic markers associated with scrotal hernias.

Additional objects and advantages of the invention will be set forth in part in the description that follows, and in part will be obvious from the description, or may be learned by the practice of the invention. The objects and advantages of the invention will be attained by means of the instrumentality's and combinations particularly pointed out in the appended claims.

### SUMMARY OF THE INVENTION

To achieve the objects and in accordance with the purpose of the invention, as embodied and broadly described herein, the present invention provides a method for screening animals for scrotal hernias.

Hernias are the result of asynchrony of the timing of the closure of the inguinal canal in prenatal/postnatal development. If the canal closes too late, then inguinal, or scrotal hernias can develop. If the canal closes too early, the testes will fail to descend into the scrotum causing a condition known as cryptorchidism. Pigs affected by cryptorchidism are known as ridglings or rigs. As used herein, the term “scrotal hernia” is intended to refer to any condition resulting from the untimely closure of the inguinal canal, including, but not limited to, inguinal hernia, scrotal hernia, and cryptorchidism.

Thus, the present invention provides a method for screening pigs for scrotal hernias, which method comprises the steps: 1) obtaining a sample of tissue or genomic DNA from an animal; and 2) analyzing the mRNA or genomic DNA obtained in 1) to determine which allele(s) is/are present. Briefly, the sample of genetic material is analyzed to determine the presence or absence of a particular allele that is correlated with a desirable or undesirable trait, or one which is linked thereto. Also included are haplotype data which allows for a series of polymorphisms in the MIS and GPX4A genes to be combined in a selection or identification protocol to maximize the benefits of each of the markers.

As is well known to those of skill in the art, a variety of techniques may be utilized when comparing nucleic acid molecules for sequence differences. These include by way of example, restriction fragment length polymorphism analysis, heteroduplex analysis, single strand conformation polymorphism analysis, single base extension, mass spectrometry, denaturing gradient electrophoresis, temperature gradient electrophoresis, DNA sequencing, and oligo ligation assay (ligase chain reaction).

In one embodiment, the polymorphism is a restriction fragment length polymorphism and the assay comprises identifying the gene from isolated genetic material; exposing the gene to a restriction enzyme that yields restriction fragments of the gene of varying length; separating the restriction fragments to form a restriction pattern, such as by electrophoresis or HPLC separation; and comparing the resulting restriction fragment pattern from an animal gene that is either known to have or not to have the undesirable markers. If an animal tests positive for the markers (or alleles), such animal can be considered for exclusion in the breeding program. If the animal does not test positive for the markers, the animal can be considered for inclusion in the breeding program.

In a most preferred embodiment, the gene, or a fragment thereof, is isolated by the use of primers and DNA polymerase to amplify a specific region of the gene which contains the polymorphism or a polymorphism linked thereto. Next, the amplified region is either directly separated or sequenced or is digested with a restriction enzyme and fragments are again separated. Visualization of the separated fragments, or RFLP pattern, is by simple staining of the fragments, or by labeling the primers or the nucleotide triphosphates used in amplification.

In another embodiment, the invention comprises a method for identifying a genetic marker or markers associated with scrotal hernias. Animals with high and low estimated breeding values for scrotal hernia are obtained, and used to look for polymorphisms in the MIS and GPX4A genes. A polymorphism in the gene of each animal is identified and associated with the undesirable trait. Preferably, PCR-RFLP analysis is used to determine the polymorphism.

It is also possible to establish linkage between specific alleles of alternative DNA markers and alleles of DNA markers known to be associated with a particular gene which have previously been shown to be associated with a particular trait. Thus, in the present situation, taking a particular gene, it would be possible, at least in the short term, to select for pigs, or other animals, unlikely to develop and/or produce offspring with scrotal hernias, or alternatively, against pigs likely to develop and/or produce offspring with scrotal hernias, indirectly, by selecting for certain alleles of a particular gene associated with the marker alleles through the selection of specific linked alleles of alternative chromosome markers. Markers and genes known to be linked to MIS and GPX4A include the microsatellite markers SW240, SW1686, SW1564, SW747, S0091, SWR1342, SW776, and SO226, and the genes CGRP, FSHb, INSL3, PDE4A, RSTN, and CAST (see FIG. 1).

The invention further comprises a kit for evaluating a sample of DNA for the presence in genetic material of an undesirable genetic marker located in the gene indicative of a heritable trait of predisposition to produce offspring with scrotal hernias. At a minimum, the kit is a container with one or more reagents that identify a polymorphism in the porcine MIS or GPX4A genes. Preferably, the reagent is a set of oligonucleotide primers capable of amplifying a fragment of the selected gene that contains a polymorphism. Preferably, the kit further contains a restriction enzyme that cleaves the gene in at least one place, allowing for separation of fragments and detection of polymorphic loci.

### BRIEF DESCRIPTION OF THE FIGURES

FIG. 1 is the chromosome 2 linkage map for hernia mapping. CM=centiMorgan. Genes and microsatellites order was estimated using CRIMAP and integrating information from USDA-MARC.2, PiGMaP.1.2, RH SSC2 (Rattink et al., Mamm. Genome 12(5):366-70 (2001)).

FIG. 2 is a summary of the allelic frequency differences for candidate genes on chromosome 2. Twenty (20) high and twenty (20) low for SH EBV were selected from farm A and B. Candidate genes were selected and genotypes and alleles frequency differences between the high and the low tails were calculated. FIG. 2 shows the genes and the P values (the two farms are combined). Many markers show significant differences between the high the low pools, confirming what was found in the QTL mapping study (see FIG. 3), that these two chromosomal regions are playing a major role in controlling hernia in pigs.

FIG. 3 is a QTL map (log likelihood) of a region of chromosome 2 for scrotal hernia in pigs. Two QTL peaks are found on SSC2 by affected sibpair analysis. The Relative Risk (RR) due to these loci was calculated based on the observed segregation ratios of parent alleles. Relative risk was found to be 1.30 and 1.18 for QTL at 7 and 36 cM respectively. About ⅓ of the relative risk is explained by the two QTLs on chromosome 2.

FIG. 4 is a representation of the expected band sizes following amplification of genomic DNA using MIS-specific primers (forward primer=5′-GGACTCCACCTCTGCCTTCCTC-3′ (SEQ ID NO:10); reverse primer=5′-GGAACTTCAGCAAGGGTGTTGG-3′ (SEQ ID NO:11); PCR length=1200 bp), then digestion of the amplification product with HaeIII.

FIG. 5 is a representation of the expected band sizes following amplification of genomic DNA using MIS-specific primers (forward primer=5′-CCAGCAACAGACAAATACACG-3′ (SEQ ID NO:12); reverse primer=5′-GCTCCAGGTGCCAAACCTGC-3′ (SEQ ID NO:13); PCR length=˜200 bp), then digestion of the amplification product with PmlI. The 20 bp band is not usually seen.

FIG. 6 is a representation of the expected band sizes following amplification of genomic DNA using MIS-specific primers (forward primer=5′-GGATGTTTAGGGCAGCAGGCAA-3′ (SEQ ID NO:14); reverse primer=5′-GCGGCGTCGCAGGGTCAGA-3′ (SEQ ID NO:15); PCR length=˜200 bp), then digestion of the amplification product with BsaJI.

FIG. 7 is a representation of the expected band sizes following amplification of genomic DNA using MIS-specific primers (forward primer=5′-CTGCGACGCCGCGGAAAT-3′ (SEQ ID NO:16); reverse primer=5′-GATGGAGGCAGGAGCTGGCTCA-3′ (SEQ ID NO:17); PCR length=123), then digestion of the amplification product with ScrfI.

FIGS. 8A and 8B are representations of the expected band sizes following amplification of genomic DNA using GPX4A-specific primers (forward primer=5′-CAGCTGCCACGGGATTACTGTT-3′ (SEQ ID NO:23); reverse primer=5′-CCCCCACCCATCACTCCATT-3′ (SEQ ID NO:24); PCR length=˜160 bp), then digestion of the amplification product with, respectively, MseI (A) and AvaI (B). In the MseI digestion, the 21 bp band is not seen.

FIG. 9 is a representation of the expected band sizes following amplification of genomic DNA using FSHb-specific primers (forward primer=5′-CCT TTA AGA CAG TCA ATG GCA A-3′ (SEQ ID NO:36); reverse primer=5′-AGT GGT TTT TCC TTC CTT TTC C-3′ (SEQ ID NO:37).

FIG. 10 shows EBV data set used to demonstrate the advantage of combining two SNPs (one SNP per QTL) on hernia incidences. The two SNPs that were used are MIS/HaeIII and FSHb. The association between EBV (multiplied by 1000) and number of copies of the good alleles (“1” for MIS and “2” for FSHb). Each dot is labeled by the genotype and number of animals within that genotype.

FIG. 11 shows the New-Sires data set used to demonstrate the advantage of combining two SNPs on hernia incidences. The association between % and progeny hernia incidence of the good alleles (“1” for MIS and “2” for FSHb). Each dot is labeled by the genotype and number of animals within that genotype.

FIG. 12 shows that the EBV and % hernia results are in agreement.

FIG. 13 shows the changes in genotype frequency of MIS/HaeIII and FSHb over time. In FIG. 13A, for MIS/HaeIII, the “11” is the good genotype. In FIG. 13B, for FSHb, the “22” is the good genotype. Unlike MIS, the frequency of the good genotype looks constant. This may be due to the fact that the good allele is already in high frequency.

FIG. 14 shows the change over the last 4 years in the relative proportion of the MIS/HaeIII-FSHb genotype combinations at farm A and B. The 9 genotype classes are ranked from good (top) to bad (bottom).

### DETAILED DESCRIPTION OF THE INVENTION

Reference will now be made in detail to the presently preferred embodiments of the invention, which together with the following examples, serve to explain the principles of the invention. All references cited herein are hereby expressly incorporated by reference.

As used herein, the term “intron” is intended to encompass any non-coding sequence occurring in a given gene. Thus, “intron” encompasses any non-coding sequence occurring between exons in a given gene, as those exons are defined by, for example, BLAST analysis.

The invention relates to the identification of quantitative trait loci (QTL) for scrotal hernias. It provides a method of screening animals to determine those more or less likely to develop and/or produce offspring with scrotal hernias by identifying the presence or absence of a polymorphism in certain genes that are correlated with these traits.

Thus, the invention relates to genetic markers and methods of identifying those markers in a pig or other animal of a particular breed, strain, population, or group, whereby an animal has scrotal hernias below the mean for that particular breed, strain, population, or group.

The marker may be identified by any method known to one of ordinary skill in the art which identifies the presence or absence of the particular allele or marker, including, for example, single-strand conformation polymorphism analysis (SSCP), base excision sequence scanning (BESS), RFLP analysis, heteroduplex analysis, denaturing gradient gel electrophoresis, allelic PCR, temperature gradient electrophoresis, ligase chain reaction, direct sequencing, single base extension, mass spectrometry, nucleic acid hybridization, and micro-array-type detection of the MIS and GPX4A genes, or other linked sequences, and examination for a polymorphic site. Yet another technique includes an Invader Assay which includes isothermic amplification that relies on a catalytic release of fluorescence. See Third Wave Technology at www.twt.com. All of these techniques are intended to be within the scope of the invention. A brief description of these techniques follows.

Isolation and Amplification of Nucleic Acid

Samples of patient, proband, test subject, or family member genomic DNA are isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W.H. Freeman & Co. New York (1992). Genomic DNA can also be isolated from cultured primary or secondary cell cultures or from transformed cell lines derived from any of the aforementioned tissue samples.

Samples of patient, proband, test subject or family member RNA can also be used. RNA can be isolated from tissues expressing the MIS and GPX4A genes as described in Sambrook et al., supra. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

PCR Amplification

The most common means for amplification is polymerase chain reaction (PCR), as described in U.S. Pat. Nos. 4,683,195, 4,683,202, 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on sterile Ficoll-Hypaque gradient by standard procedures. Interphase cells are collected and washed three times in sterile phosphate buffered saline before. DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl 1.5 mM MgCl₂, 0.5% Tween 20, 0.5% NP40 supplemented with 100 μg/ml of proteinase K. After incubating at 56° C. for 2 hr. the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N.H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference.

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification; the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both polymorphic forms of the allele, but which at high stringency, bind to the allele to which they correspond. Alternatively, stringency conditions can be devised in which an essentially binary response is obtained, i.e., an ASO corresponding to a variant form of the target gene will hybridize to that allele, and not to the wildtype or “consensus” allele.

Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (TM). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, Principles and Applications for DNA Amplification, W.H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tₘ of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tm's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis

Target sequences or alleles at the MIS and GPX4A loci can be differentiated using single-strand conformation polymorphism analysis, which identifies base differences by alteration in electrophoretic migration of single stranded PCR products, as described in Orita et al., Proc. Nat. Acad. Sci. 85:2766-2770 (1989). Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from a patient and an affected family member may be used to generate mismatch free heterohybrid DNA duplexes. As used herein, “heterohybrid” means a DNA duplex strand comprising one strand of DNA from one person, usually the patient, and a second DNA strand from another person, usually an affected or unaffected family member. Positive selection for heterohybrids free of mismatches allows determination of small insertions, deletions or other polymorphisms that may be associated with alterations in androgen metabolism.

Non-PCR Based DNA Diagnostics

The identification of a DNA sequence linked to MIS and/or GPX4A can be made without an amplification step, based on polymorphisms including restriction fragment length polymorphisms in a subject and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with 32P or 35S. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and e, e′, 5,5′-5354amethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the porcine chromosome where MIS and GPX4A resides, and thus defining genetic markers linked to MIS and GPX4A, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

Although the methods described herein may be in terms of the use of a single restriction enzyme and a single set of primers, the methods are not so limited. One or more additional restriction enzymes and/or probes and/or primers can be used, if desired. Additional enzymes, constructed probes and primers can be determined through routine experimentation, combined with the teachings provided and incorporated herein.

Genetic markers for genes are determined as follows. Male and female animals of the same breed or breed cross or derived from similar genetic lineages are mated. The offspring with the undesirable trait are determined. RFLP analysis of the parental DNA is conducted as discussed above in order to determine polymorphisms in the selected gene of each animal. The polymorphisms are associated with the traits.

When this analysis is conducted and the polymorphism is determined by RFLP or other analysis, amplification primers may be designed using analogous human or other closely related animal known sequences. The sequences of many of the genes have high homology. Primers may also be designed using known gene sequences as exemplified in Genbank or even designed from sequences obtained from linkage data from closely surrounding genes. According to the invention, sets of primers have been selected which identify regions in polymorphic genes. The polymorphic fragments have been shown to be alleles, and several were shown to be associated with scrotal hernias.

The reagents suitable for applying the methods of the invention may be packaged into convenient kits. The kits provide the necessary materials, packaged into suitable containers. At a minimum, the kit contains a reagent that identifies a polymorphism in the selected gene that is associated with a trait. Preferably, the reagent is a PCR set (a set of primers, DNA polymerase and 4 nucleoside triphosphates) that hybridize with the gene or a fragment thereof. Preferably, the PCR set is included in the kit. Preferably, the kit further comprises additional means, such as reagents, for detecting or measuring the detectable entity or providing a control. Other reagents used for hybridization, prehybridization, DNA extraction, visualization etc. may also be included, if desired.

The methods and materials of the invention may also be used more generally to evaluate animal DNA, to identify analogous polymorphisms in animals other than those for whom sequences have been disclosed herein, genetically type individual animals, and detect genetic differences in animals.

In particular, a sample of genomic DNA may be evaluated by reference to one or more controls to determine if a polymorphism in the gene is present. Preferably, RFLP analysis is performed with respect to the gene, and the results are compared with a control. The control is the result of a RFLP analysis of the gene of a different animal where the polymorphism of the gene is known. Similarly, the genotype of an animal may be determined by obtaining a sample of its mRNA or genomic DNA, conducting RFLP analysis of the gene in the DNA, and comparing the results with a control. Again, the control is the result of RFLP analysis of the same gene of a different animal. The results genetically type the animal by specifying the polymorphism in its selected gene. Finally, genetic differences among animals can be detected by obtaining samples of the mRNA or genomic DNA from at least two animals, identifying the presence or absence of a polymorphism in the gene, and comparing the results.

These assays are useful for identifying the genetic markers relating to scrotal hernias, as discussed above, for identifying other polymorphisms in the gene that may be correlated with other characteristics, and for the general scientific analysis of genotypes and phenotypes.

The genetic markers, methods, and kits of the invention are also useful in a breeding program to reduce the incidence of scrotal hernias in a breed, line, or population of animals. Continuous selection and breeding of animals that are at least heterozygous and preferably homozygous for a polymorphism associated with a beneficial trait would lead to a breed, line, or population having lower numbers of scrotal hernias in the males of this breed or line. Thus, the markers are selection tools.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. In this case the Reference MIS or GPX4A sequences. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Clayerie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involved no more than routine optimization of parameters using the teachings herein.

It is also possible to establish linkage between specific alleles of alternative DNA markers and alleles of DNA markers known to be associated with a particular gene (e.g., the MIS, GPX4A, and FSHb genes discussed herein), which have previously been shown to be associated with a particular trait. Thus, in the present situation, taking the MIS and GPX4A genes, it would be possible, at least in the short term, to select for pigs more or less likely to develop and/or produce offspring with scrotal hernias indirectly, by selecting for certain alleles of a MIS or GPX4A-associated marker through the selection of specific alleles of alternative chromosome markers. As used herein the term “genetic marker” shall include not only the polymorphism disclosed by any means of assaying for the protein changes associated with the polymorphism, be they linked markers, use of microsatellites, or even other means of assaying for the causative protein changes indicated by the marker and the use of the same to influence the incidence of scrotal hernias. Markers and genes known to be linked to MIS, GPX4A, and FSHb include the microsatellite markers SW240, SW1686, SW1564, SW747, S0091, SWR1342, SW776, and SO₂₂₆, and the genes CGRP, INSL3, PDE4A, RSTN, and CAST.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism, for example http://darwin.bio.geneseo.edu which can give restriction enzymes upon analysis of a sequence and the polymorphism to be identified. In fact as disclosed in the teachings herein there are numerous ways of identifying a particular polymorphism or allele with alternate methods which may not even include a restriction enzyme, but which assay for the same genetic or proteomic alternative form.

In yet another embodiment of this invention novel porcine nucleotide sequences have been identified and are disclosed which encode porcine MIS and GPX4A. The cDNA of the porcine MIS and GPX4A genes as well as some intronic DNA sequences are disclosed. These sequences may be used for the design of primers to assay for the SNP's of the invention or for production of recombinant MIS or GPX4A. The invention is intended to include these sequences as well as all conservatively modified variants thereof as well as those sequences which will hybridize under conditions of high stringency to the sequences disclosed. The term MIS or GPX4A as used herein shall be interpreted to include these conservatively modified variants as well as those hybridized sequences.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention. When it is desired to alter the amino acid sequence of a polypeptide to create an equivalent, or even an improved variant or portion of a polypeptide of the invention, one skilled in the art will typically change one or more of the codons of the encoding DNA sequence according to Table 1.

TABLE 1

| | |
|---|---|
| Amino Acids | Codons |

| | | | |
|---|---|---|---|
| Alanine | Ala | A | GCA GCG GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Me | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W.H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. The term “stringent conditions”or “stringent hybridization conditions” includes reference to conditions under which a probe will hybridize to its target sequence, to a detectably greater degree than to other sequences (e.g., at least 2-fold over background). Stringent conditions are sequence-dependent and be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C. for about ten hours and preferably overnight, and a wash in 1× to 2×SSC (20×SSC=3.0 M NaCl/0.3 M trisodium citrate) at 50 to 55° C. for about 15 minutes. Exemplary moderate stringency conditions include hybridization in 40 to 45% formamide, 1 M NaCl, 1% SDS at 37° C. for at least 10 hours and preferably overnight, and a wash in 0.5× to 1×SSC at 55 to 50° C. for about about 15 minutes. Exemplary high stringency conditions include hybridization in 50% formamide, 1 M NaCl, 1% SDS at 37° C. for at least 10 hours and preferably overnight, and a wash in 0.1×SSC at 60 to 65° C. for about 15 minutes.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acids Probes, Part I, Chapter 2, Ausubel, et al., Eds., Greene Publishing and Wiley-Interscience, New York (1995).

The examples and methods herein disclose certain genes which have been identified to have a polymorphism which is associated either positively or negatively with a beneficial trait that will have an effect on the incidence of scrotal hernias. The identification of the existence of a polymorphism within a gene is often made by a single base alternative that results in a restriction site in certain allelic forms. A certain allele, however, as demonstrated and discussed herein, may have a number of base changes associated with it that could be assayed for which are indicative of the same polymorphism. Further, other genetic markers or genes may be linked to the polymorphisms disclosed herein so that assays may involve identification of other genes or gene fragments, but which ultimately rely upon genetic characterization of animals for the same polymorphism. Markers and genes known to be linked to MIS and GPX4A include the microsatellite markers SW240, SW1686, SW1564, SW747, S0091, SWR1342, SW776, and SO₂₂₆, and the genes CGRP, FSHb, INSL3, PDE4A, RSTN, and CAST. Any assay which sorts and identifies animals based upon the allelic differences disclosed herein are intended to be included within the scope of this invention.

One of skill in the art, once a polymorphism has been identified and a correlation to a particular trait established, will understand that there are many ways to genotype animals for this polymorphism. The design of such alternative tests merely represent optimization of parameters known to those of skill in the art and are intended to be within the scope of this invention as fully described herein.

### EXAMPLE

Using a candidate gene approach, the inventors have found markers in three genes (MIS, GPX4A, and FSHb) in a region of pig chromosome 2 that are associated with incidence of SH. This region of chromosome 2 has also been implicated in SH using genome scanning with microsatellites and AFLP. Use of either single markers or combinations of markers (haplotypes) from this region are useful in selecting against breeding animals with a predisposition to produce SH offspring.

Polymorphisms within the MIS gene (Mullerian inhibitory substance) were shown to have association with SH in a line of pigs. MIS maps to pig chromosome 2 and QTL scans of this chromosome using AFLP and microsatellite markers as well as SNPs also indicated a region associated with SH between SW240 and SO226. Several further candidate genes within this interval were investigated including FSHb (follicle stimulating hormone b), CGRP (calcitonin gene-related peptide), INSL3 (Insulin-like 3), PDE4A (phosphodiesterase 4A), GPX4A (phospholipid hydroperoxide glutathione peroxidase 4A), RSTN (resistin), and CAST (calpastatin). An association between SH associations between markers in GPX4A were also seen.

Experimental Approaches

Animals from a line of pigs were ranked for predisposition to produce SH offspring using estimated breeding values (EBV). 20 high SHEBV and 20 low SHEBV animals from each of two farms (A and B) were used to look for single nucleotide polymorphisms (SNPs) in candidate genes. SNP discovery in candidate genes compared the DNA sequence of high vs. low EBV pools (four animals/pool). Sequence was performed using ABI 3100. Identified SNPs were validated by PCR-RFLP wherever possible. Allelic frequencies of markers were calculated and contrasted between high vs. low EBV animals.

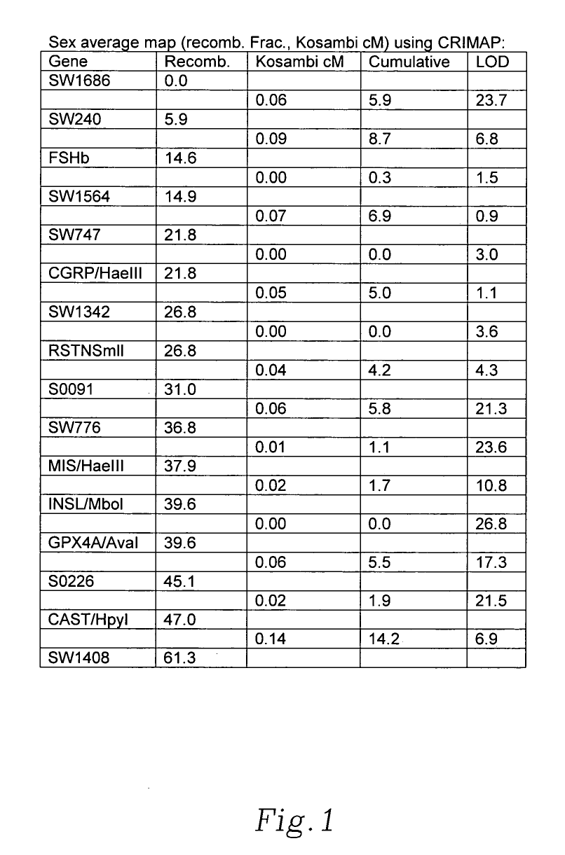

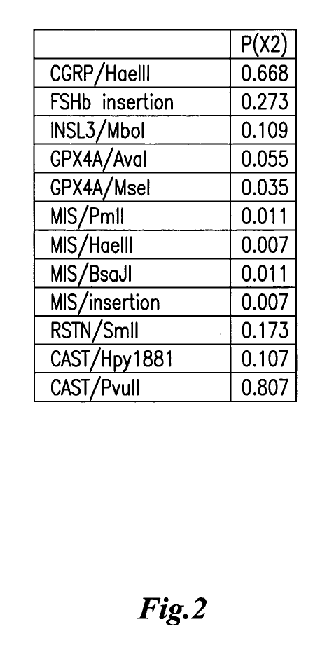

Animals from a commercial unit were used for genome scanning to define an SH QTL on pig chromosome 2 using SNPs in candidate genes and microsatellite markers (FIGS. 1 and 2), and affected sib pair methodology (Kruglyak and Lander, 1995; Am J Hum Genet. 57:439-54).

Results

A summary of the MIS and GPX4A SNPs is shown in Tables 2 and 3, respectively. The sequence of the MIS gene is shown in Table 4, and the MIS SNP test protocols are shown in Table 5. The sequence of the GPX4A gene is shown in Table 6, and the GPX4A SNP test protocol is shown in Table 7.

A summary of the FSHb SNP is shown in Table 13. The sequence of the FSHb gene is shown in Table 13, and the FSHb SNP test protocaol is shown in Table 14.

The difference of allelic frequencies of candidate genes comparing animals with high vs. low hernia EBV indicates that the region containing MIS and GPX4A is associated with SH (see FIG. 2).

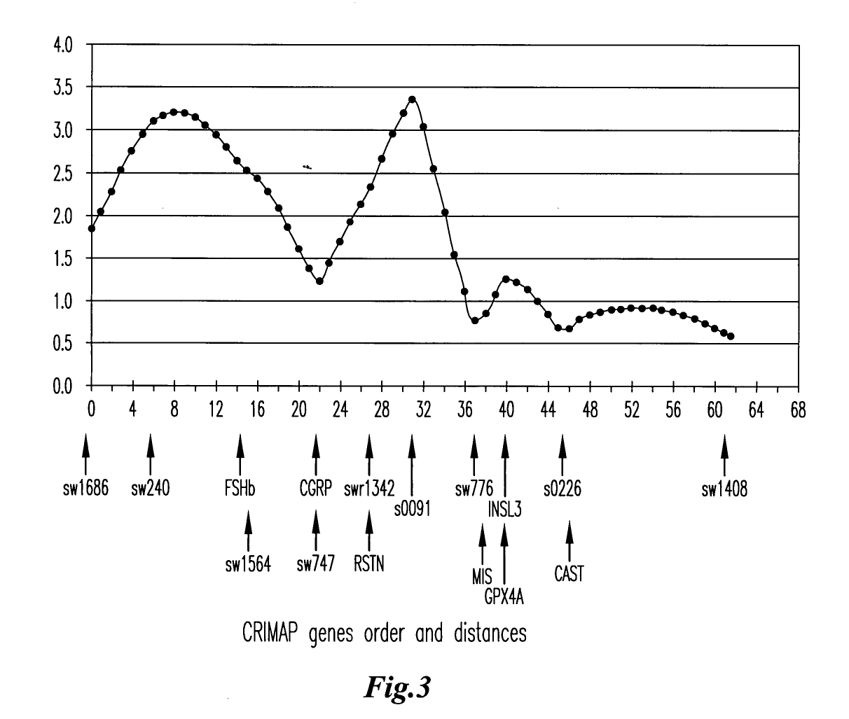

Based on both QTL mapping (see FIG. 3) and allelic frequency analysis (FIG. 2), MIS may be either itself a major gene for susceptibility to SH or is closely linked to such a major gene.

A haplotype analysis of SNPs in GPX4A and MIS was carried out using the SHEBV animals to see if the discrimination between animals with high or low SHEBV could be improved (see Tables 8). The analysis showed that the GPX4A-MIS haplotype was significantly associated with scrotal hernia estimated breeding value. However, MIS alone showed a greater association with SHEBV than the GPX4A-MIS haplotype.

TABLE 2
Summary of MIS SNPs

| | | | | Association |
|---|---|---|---|---|
| | | | | with hernia |
| | | | Change | (avg. of |
| | | | of | Farms A |
| Gene | Region | Type of SNP | nucleotide | and B) |
| MIS | Intron 1 | RFLP (HaeIII) | A/G | Yes |
| MIS | Intron 1 | RFLP (PmlI) | C/T | Yes |
| MIS | Exon 3 (silent | RFLP (BsaJI) | C/T | Yes |
| | mutation) | | | |
| MIS | Intron 3 | Insertion/deletion | ACCAC | Yes |

TABLE 3
Summary of GPX4A SNPs

| | | | | Association |
|---|---|---|---|---|
| | | | | with hernia |
| | | | Change of | (avg. of Farms |
| Gene | Region | Type of SNP | nucleotide | A and B) |
| GPX4A | Intron 4 | RFLP (MseI) | G/A | Yes in A |
| GPX4A | Intron 5 | RFLP (AvaI) | C/T | Yes in A |

TABLE 4

Porcine MIS gene sequence (coding and non-
coding regions)

5′untranslated region (SEQ ID NO:1)
GGACTCCACCTCTGCCTTCCTCCAGCCACCCCTACCCCCACCACAAGCTG
TTGACAGTCTGGCCATTCACTCCCTGCTCACATTYCCACTCCCGGTTCTA
AAAGGGGAAAACTTGTCAAGGACAGTCTTGACAAATGGGTCACAGGCCAC
CCTTCTATCACTAGTAAGGAGATAGGCAGTCAGGTTGGAACAGAAGAGGT
TTTGAGAAGCCTGCTGGCTTGCCCAGGCTCACAGCAGGCACCGGCCTCCA
AGGTCACATCCCAGAAGGAGATAGGGGCTGGCCTCCCACACCCACATTCC
TGCTCCCCCATATAAGCCAGGGCAGCCCAGCCCCTCAAAGTGCCAGG
Exon 1 (SEQ ID NO:2)
ATGCAGGGTCCTTCTCTCTCTCAGCTGGTCCTGGTGCTGGCAGCAATGGG
GGCCCTGCTGGAGGCTGGGACCCCCAGAGAAGAGGTCTCCAGCACCCCAG
CTTTGCCCAGGGAGCCAGCCACAGGCACCGAGGGGCTCATCTTCCACTGG
GACTGGAACTGGCCGCCCCCTGGTGCCTGGCCCCTGGGTGGCCCTCAGGA
CCCCCTGTGCCTAGTGACCCTGAATGGAGACCCTGGCAATGGGAGCAGTC
CTTTTCTGTGGGTGGTGGGGACTCTAAGCAGTTATGAGCAGGCCTTCCTG
GAGGCTGTGCGGCATGCCCGCTGGGGTCCCCAAGACCTGGCCAACTTTGG
GCTCTGCCCTCCCAGCCTCAGGCAGGCTGCCCTGCCCCTTTTGCAGCAGC
TGCAGGCATGGCTGGGGGAGCCCAGGGGGCAGCGACTGGTGGTCCTGCAC
CTGGAGGAA
Intron 1 (SEQ ID NO:3)
TGCCCTGCCCCTTTTGCAGCAGCTGCAGGCATGGCTGGGGGAGCCCAGGG
GGCAGCGACTGGTGGTCCTGCACCTGGAGGAAGGTACGTGGGGGCTGCAG
CGGGACCTGGTGGGTGGGCAGAGGACTGGGCTCTAGTCTCAGGATGGGAG
ACGACTGTTTCTTGCYTAGAGCCGCACCCAGCCTCCTCAGGAAGTTGAGG
CTGATGGCCAGACAGGTGGGTGACCTTATTTTGCCCTGTCTGGGAGTGCC
TCCTCCAGTACCTGGGAAGGTCCAGCAACAGACAAATACACAY¹GR²CCA
TGGACCTCAGGGACCCACTGCAGGGAAKGGCTTCCCTCCAGGAGAGCTTC
AGACCAAGAGACCCCAAGGGCTTGGGTAACCCACAGCAGTGGGGGCAGTG
CTCCACCACCCACCCTATGCATCCCTCCTCCCAGGTTGCCTGTCCCAGGC
AGGTTTGGCACCTGGAGCCCAAGGGTATCAAGTGTCTCTCAGACACAGAG
CCGTCCCCCCACTGAGGCTCCCCCTCCTGCACAGGCGACAGGCTTTGGGG
GAGGGTCTTGGGCTTCTGTGGTTCAGGCAACTCTGTCCACTTCCCCCTTT
GTCCTGGCCACA
Exon 2 (SEQ ID NO:4)
GTGTCGTGGGAGCCAACACCCTTGCTGAAGTTCCAGGAGCCCCTACCTGG
AGAAGCCAGCCCCCTGGAGCTGGCGCTGTTGGTGTTGTATCCAGGGCCCG
GCCCAGAGGTCACTGTCACCGGGGCTGGGCTGCCCGGCGCCCAG
Intron 2 (SEQ ID NO:5)
GTACCAGAGAGGTGAATGAGGCTGTCCCTGGGCCACCAGGAGCCCTCATT
CAAGGCAAGGGCGGGATTATTGAGGGGGGGGG(GG)KTAACTGCACCTAA
CAGAAAGGCTGTGACTGTCCAAGTTGGAATTTTGCAGGGATGTTTAGGGC
AGCAGGCAAGCAGGGCTGGTGTCCCAAGGCCCCAGCAAGCCTGGCTGAGT
CCCCATCTCCACAG
Exon 3 (SEQ ID NO:6)
AGCCTCTGCCCGACY³AGGGACTCTGGCTTCCTGGCGTTGGCGGTCGACC
ACCCAGAGAGGGCCTGGCGTGGCTCTGGGCTTGCTCTGACCCTGCGACGC
CGCGGAAAT
Intron 3 (SEQ ID NO:7)
GGTAGCCCCCTCCCCCAGACTGGAGCCGGGCTGGGGCGGCTGCCCTCGGA
AACACCCCCCCC(ACCAC)⁴CCTTCCAGYCGSTGAGCCAGCTCCTGCCTCC
ATCCTCA
Exon 4 (SEQ ID NO:8)
GGTGCCTCCCTGAGCACCGCCCAGCTGCAGGCGCTGCTGTTTGGCGCCGA
CTCTCGCTGCTTCACACGGATGACCCCGGCCCTGCTCCTGTTGCCGCCGC
AGGGGCCGGTGCCGATGCCCGCACACGGCCGGGTGGACTCAATGCCATTC
CCGCAGCCCAGG
Intron 4 (SEQ ID NO:9)
CTGCGCATGAGTCAGAACTTGGGGGCGCAGGGACGTGGGGGCAGCGCAGG
CTTGTGCCCTCACGTCCCCGCGCTCCGCCGTCCAGGCTGTCCCCAGAGCC
C

¹=MIS/PmlI SNP

²=MIS/HaeIII SNP

³=MIS/BsaJI SNP

⁴=MIS insertion/deletion

Y, R, K & S=other SNPs not studied further

(GG)=small insertion/deletion, not studied further

Table 5. MIS SNP Test Protocols

MIS/HaeIII Protocol

| | | | |
|---|---|---|---|
| Forward primer: | MIS5_2-2F | 5′-GGACTCCACCTCTGCCTTCCTC-3′ | (SEQ ID NO:10) |
| Reverse primer: | MIS5_2-2R | 5′-GGAACTTCAGCAAGGGTGTTGG-3′ | (SEQ ID NO:11) |

PCR length is ˜1200 bp

PCR reagents:

| | |
|---|---|
| 10 × PCR Buffer II | 1.0 μl |
| 2 mM dNTP's | 1.0 μl |
| 25 mM MgCl₂ | 0.6 μl |
| MIS5_2-2F (5 μM) | 1.0 μl |
| MIS5_2-2R (5 μM) | 1.0 μl |
| Amplitaq Gold | 0.1 μl |
| QH₂0 | 4.3 μl |
| Genomic DNA | 1.0 μl |

PCR Program Using PE9700:

94° C. 1 Mins

95° C. 5 min→61° C. 45 Secs×38→72° C. 7 min→4° C. ∞

72° C. 1 Mins 20 Secs

(9600 Ramp)

Digestion:

| | |
|---|---|
| PCR Product | 10.0 μl |
| 10 × NE Buffer 2 | 1.5 μl |
| 100 × BSA | 0.15 μl |
| Rediload | 0.5 μl |
| HaeIII (1O u/μl) | 0.3 μl |
| ddH₂0 | 2.55 μl |

Digest at 37° C. for 4 Hours

Load and run on 3% NuMe Agarose at 150 volts

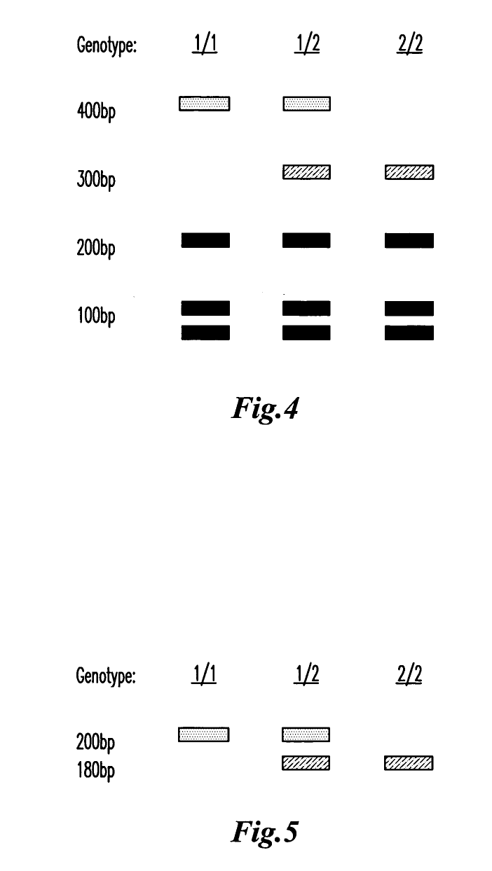

FIG. 4 shows the band sizes expected.

MIS/PmlI Protocol

| | | | |
|---|---|---|---|
| Forward primer: | MIS-SNP4F | 5′-CCAGCAACAGACAAATACACG-3′ | (SEQ ID NO:12) |
| Reverse primer: | MIS-SNP4R-1 | 5′-GCTCCAGGTGCCAAACCTGC-3′ | (SEQ ID NO: 13) |

PCR length is ˜200 bp

PCR reagents:

| | |
|---|---|
| 10 × PCR Buffer II | 1.0 μl |
| 2 mM dNTP's | 1.0 μl |
| 25 mM MgCl₂ | 0.6 μl |
| MIS-SNP4F (5 μM) | 1.0 μl |
| MIS-SNP4R-1 (5 μM) | 1.0 μl |
| Amplitaq Gold | 0.1 μl |
| QH₂0 | 4.3 μl |
| Genomic DNA | 1.0 μl |

PCR program using PE9700:

94° C. 1 Mins

95° C. 5 min→60° C. 20 Secs×35→72° C. 7 min→4° C. ∞

72° C. 20 Secs

(9600 Ramp)

Digestion:

| | |
|---|---|
| PCR Product | 10.0 μl |
| 10 × NE Buffer 1 | 1.5 μl |
| 100 × BSA | 0.15 μl |
| Rediload | 0.5 μl |
| Pm1I (20 u/μl) | 0.2 μl |
| ddH₂0 | 2.65 μl |

Digest at 37° C. for 4 Hours

Load and run on 3% NuMe Agarose at 150 volts

FIG. 5 shows the band sizes expected (the 20 bp is not usually seen).

MIS/BsaJI Protocol

| | | | |
|---|---|---|---|
| Forward primer: | MISintr2-2F | 5′-GGATGTTTAGGGCAGCAGGCAA-3′ | (SEQ ID NO:14) |
| Reverse primer: | MISintr2SNP-R | 5′-GCGGCGTCGCAGGGTCAGA-3′ | (SEQ ID NO:15) |

PCR length is ˜200 bp

PCR reagents:

| | |
|---|---|
| 10 × PCR Buffer II | 1.0 μl |
| 2 mM dNTP's | 1.0 μl |
| 25 mM MgCl₂ | 0.4 μl |
| MISintr2-2F (5 μM) | 2.0 μl |
| MISintr2SNP-R (5 μM) | 1.0 μl |
| Amplitaq Gold | 0.1 μl |
| QH₂0 | 4.5 μl |
| Genomic DNA | 1.0 μl |

PCR program using PE9700:

94° C. 30 Secs

95° C. 5 min→61° C. 30 Secs×35→72° C. 7 min→4° C. ∞

72° C. 25 Secs

(9600 Ramp)

Digestion:
BsaJI

| | |
|---|---|
| PCR Product | 10.0 μl |
| 1O × NEB buffer 2 | 1.5 μl |
| 100 × BSA | 0.15 μl |
| Rediload | 0.5 μl |
| BsaJI (2.5 u/μl) | 0.6 μl |
| Rediload | 2.25 μl |

Digest at 60° C. for 4 Hours (BsaJI)

Load and run on 3% NuMe Agarose at 150 volts

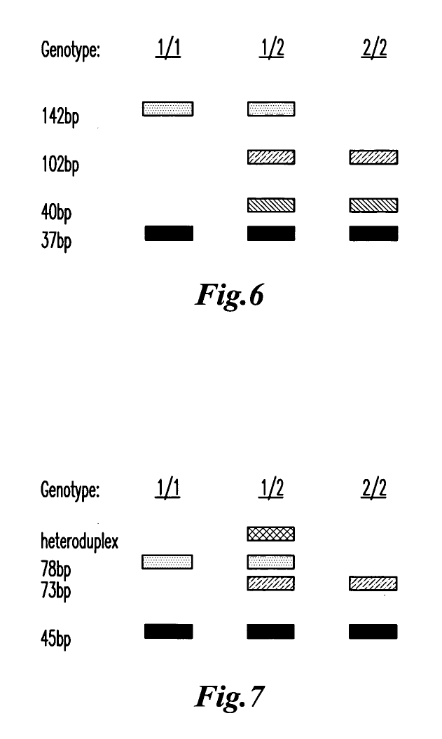

FIG. 6 shows the band sizes expected for BsaJI digestion.

MIS/Insertion Protocol

| | | | |
|---|---|---|---|
| Forward primer: | MIS insertF | 5′-CTGCGACGCCGCGGAAAT-3′ | (SEQ ID NO:16) |
| Reverse primer: | MIS intr3-R | 5′-GATGGAGGCAGGAGCTGGCTCA-3′ | (SEQ ID NO: 17) |

PCR length is ˜123 bp

PCR reagents:

| | |
|---|---|
| 10 × PCR Buffer II | 1.0 μl |
| 2 mM dNTP's | 1.0 μl |
| 25 mM MgCl₂ | 0.6 μl |
| MIS insertF (5 μM) | 1.0 μl |
| MIS intr3-R (5 μM) | 1.0 μl |
| Amplitaq Gold | 0.1 μl |
| QH₂0 | 4.3 μl |
| Genomic DNA | 1.0 μl |

PCR program using PE9700:

94° C. 30 Secs

95° C. 5 min→+61° C. 30 Secs×35→72° C. 7 min→4° C. ∞

72° C. 15 Secs

(9600 Ramp)

Digestion:

| | |
|---|---|
| PCR Product | 10.0 μl |
| 10 × NE Buffer 4 | 1.5 μl |
| 100 × BSA | 0.15 μl |
| Rediload | 0.5 μl |
| ScrFI(10 u/μl) | 0.3 μl |
| ddH₂0 | 2.55 μl |

Digest at 37° C. for 4 Hours

Load and run on 4% NuMe Agarose at 150 volts

FIG. 7 shows the band sizes expected.

table 6
Porcine GPX4 gene sequence

Exon 4 (SEQ ID NO:18)
GAGCCAGGGAGTGATGCTGAGATCAAAGAATTTGCTGCTGGCTACAACGT
CAAATTTGATATGTTCAGCAAGATCTGTGTGAATGGGGACGATGCCCACC
CTCTGTGGAAGTGGATGAAAGTCCAGCCCAAGGGGAGGGGCATGCTGGGA
AA
Intron 4 (SEQ ID NO:19)
GTGAGTTGGGGGGCTGGGGTGAGAGTGGAGGGCAGTGGGGATCTGCAGCT
GCCACGGGATTACTGATR¹ACACATTTCTTTTTGCAG
Exon 5 (SEQ ID NO:20)
TGCTATCAAATGGAACTTTACCAAG
Intron 5 (SEQ ID NO:21)
GTAAGGGGGTGCTGAGGGCCY²GGGGGGTGCCCTCAGTCACCCTGGTGCC
ACTTCTAGGGTCTCCACCTGACCTAAATGGAGTGATGGGTGGGGGCCGCT
TGCTTGCTTGCCCCAGTCCCACCACGGTGGCCTTCTGTCCCTGACACCAC
CTGTCCTGCAG
Exon 6 (SEQ ID NO: 22)
TTCCTCATTGATAAGAACGGCTGTGTGGTGAAGCGGTACGGTCCCATGGA
AGAGCCCCAG

¹=GPX4A/MseI SNP

²=GPX4A/AvaI SNP

Table 7. GPX4A SNP Test Protocol

GPX4A/MseI and GPX4A/AvaI Protocol

| | | | |
|---|---|---|---|
| Forward primer: | GPX4_6SNP1F | 5′-CAGCTGCCACGGGATTACTGTT-3′ | (SEQ ID NO:23) |
| Reverse primer: | GPX4_6SNP1R | 5′-CCCCCACCCATCACTCCATT-3′ | (SEQ ID NO:24) |

PCR length is ˜160 bp

PCR reagents:

| | |
|---|---|
| 10 × PCR Buffer II | 2.0 μl |
| 2 mM dNTP's | 2.0 μl |
| 25 mM MgCl₂ | 1.2 μl |
| GPX4_6 SNP1F (5 μM) | 2.0 μl |
| GPX4 6 SNPIR (5 μM) | 2.0 μl |
| Amplitaq Gold | 0.2 μl |
| QH₂0 | 8.6 μl |
| Genomic DNA | 2.0 μl |

PCR program using PE9700:

94° C. 45 Secs

95° C. 5 min→60° C. 30 Secs×35→72° C. 7 min→4° C. ∞

72° C. 20 Secs

(9600 Ramp)

Digestion:

| MseI | | AvaI | |
|---|---|---|---|
| PCR Product | 10.0 μl | PCR Product | 10.0 μl |
| 10 × NE Buffer 2 | 1.5 μl | 10 × NEB buffer 4 | 1.5 μl |
| 100 × BSA | 0.15 μl | 100 × BSA | 0.15 μl |
| Rediload | 0.5 μl | Rediload | 0.5 μl |
| MseI(10 u/μl) | 0.3 μl | AvaI (10 u/μl) | 0.3 μl |
| ddH₂0 | 2.55 μl | Rediload | 2.55 μl |

Digest at 37° C. for 4 Hours

Load and run on 3% NuMe Agarose at 150 volts

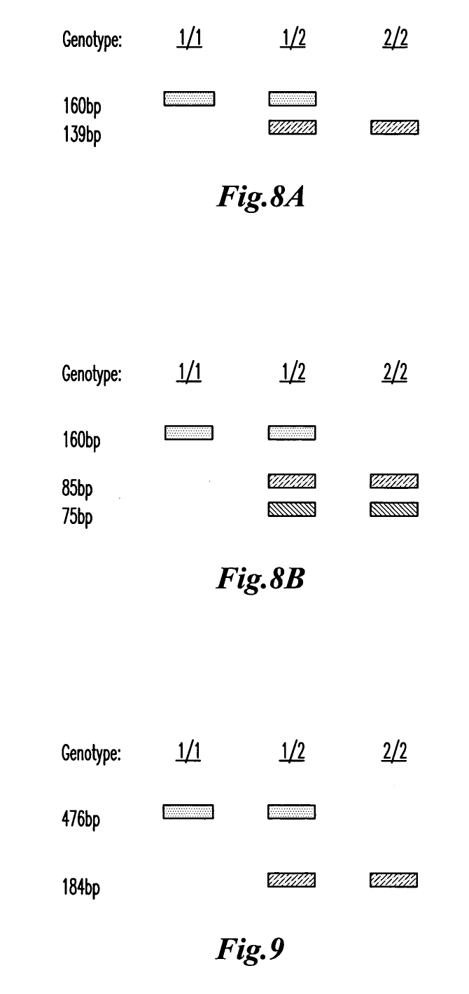

FIGS. 8A and 8B show the band sizes expected for, respectively, MseI and AvaI digestion (the 21 bp band is not seen in FIG. 9A).

TABLE 8
Preliminary results of haplotype on SSC2*

| | GPX4A-MIS | | | | |
|---|---|---|---|---|---|
| | haplotype frequency | | | | |
| | 11 | 12 | 21 | 22 | No. |
| AHEBV | 22 | 6 | 3 | 69 | 16 |
| ALEBV | 56 | 0 | 9 | 34 | 16 |
| BHEBV | 31 | 0 | 4 | 65 | 13 |
| BLEBV | 41 | 3 | 13 | 44 | 16 |
| CHEBV | 26 | 3 | 3 | 67 | 29 |
| CLEBV | 48 | 2 | 11 | 39 | 32 |

*The most informative SNP in each gene was used for haplotyping covering approximately 18 cM of chromosome 2

TABLE 9

Coding Portion of Consensus Porcine MIS
(Exon 1-Exon 2-Exon 3-Exon 4) (SEQ ID NO: 25)

ATGCAGGGTCCTTCTCTCTCTCAGCTGGTCCTGGTGCTGGCAGCAATGGG
GGCCCTGCTGGAGGCTGGGACCCCCAGAGAAGAGGTCTCCAGCACCCCAG
CTTTGCCCAGGGAGCCAGCCACAGGCACCGAGGGGCTCATCTTCCACTGG
GACTGGAACTGGCCGCCCCCTGGTGCCTGGCCCCTGGGTGGCCCTCAGGA
CCCCCTGTGCCTAGTGACCCTGAATGGAGACCCTGGCAATGGGAGCAGTC
CTTTTCTGTGGGTGGTGGGGACTCTAAGCAGTTATGAGCAGGCCTTCCTG
GAGGCTGTGCGGCATGCCCGCTGGGGTCCCCAAGACCTGGCCAACTTTGG
GCTCTGCCCTCCCAGCCTCAGGCAGGCTGCCCTGCCCCTTTTGCAGCAGC
TGCAGGCATGGCTGGGGGAGCCCAGGGGGCAGCGACTGGTGGTCCTGCAC
CTGGAGGAAGTGTCGTGGGAGCCAACACCCTTGCTGAAGTTCCAGGAGCC
CCTACCTGGAGAAGCCAGCCCCCTGGAGCTGGCGCTGTTGGTGTTGTATC
CAGGGCCCGGCCCAGAGGTCACTGTCACCGGGGCTGGGCTGCCCGGCGCC
CAGAGCCTCTGCCCGACCAGGGACTCTGGCTTCCTGGCGTTGGCGGTCGA
CCACCCAGAGAGGGCCTGGCGTGGCTCTGGGCTTGCTCTGACCCTGCGAC
GCCGCGGAAATGGTGCCTCCCTGAGCACCGCCCAGCTGCAGGCGCTGCTG
TTTGGCGCCGACTCTCGCTGCTTCACACGGATGACCCCGGCCCTGCTCCT
GTTGCCGCCGCAGGGGCCGGTGCCGATGCCCGCACACGGCCGGGTGGACT
CAATGCCATTCCCGCAGCCCAGG

TABLE 10

Coding and Non-Coding Portions (5′UTR-
Exon 1-Intron 1-Exon 2-Intron 2-Exon 3-Intron 3-
Exon 4-Intron 4) of Consensus Porcine MIS
(SEQ ID NO:26)

GGACTCCACCTCTGCCTTCCTCCAGCCACCCCTACCCCCACCACAAGCTG
TTGACAGTCTGGCCATTCACTCCCTGCTCACATTNCCACTCCCGGTTCTA
AAAGGGGAAAACTTGTCAAGGACAGTCTTGACAAATGGGTCACAGGCCAC
CCTTCTATCACTAGTAAGGAGATAGGCAGTCAGGTTGGAACAGAAGAGGT
TTTGAGAAGCCTGCTGGCTTGCCCAGGCTCACAGCAGGCACCGGCCTCCA
AGGTCACATCCCAGAAGGAGATAGGGGCTGGCCTCCCACACCCACATTCC
TGCTCCCCCATATAAGCCAGGGCAGCCCAGCCCCTCAAAGTGCCAGGATG
CAGGGTCCTTCTCTCTCTCAGCTGGTCCTGGTGCTGGCAGCAATGGGGGC
CCTGCTGGAGGCTGGGACCCCCAGAGAAGAGGTCTCCAGCACCCCAGCTT
TGCCCAGGGAGCCAGCCACAGGCACCGAGGGGCTCATCTTCCACTGGGAC
TGGAACTGGCCGCCCCCTGGTGCCTGGCCCCTGGGTGGCCCTCAGGACCC
CCTGTGCCTAGTGACCCTGAATGGAGACCCTGGCAATGGGAGCAGTCCTT
TTCTGTGGGTGGTGGGGACTCTAAGCAGTTATGAGCAGGCCTTCCTGGAG
GCTGTGCGGCATGCCCGCTGGGGTCCCCAAGACCTGGCCAACTTTGGGCT
CTGCCCTCCCAGCCTCAGGCAGGCTGCCCTGCCCCTTTTGCAGCAGCTGC
AGGCATGGCTGGGGGAGCCCAGGGGGCAGCGACTGGTGGTCCTGCACCTG
GAGGAATGCCCTGCCCCTTTTGCAGCAGCTGCAGGCATGGCTGGGGGAGC
CCAGGGGGCAGCGACTGGTGGTCCTGCACCTGGAGGAAGGTACGTGGGGG
CTGCAGCGGGACCTGGTGGGTGGGCAGAGGACTGGGCTCTAGTCTCAGGA
TGGGAGACGACTGTTTCTTGCNTAGAGCCGCACCCAGCCTCCTCAGGAAG
TTGAGGCTGATGGCCAGACAGGTGGGTGACCTTATTTTGCCCTGTCTGGG
AGTGCCTCCTCCAGTACCTGGGAAGGTCCAGCAACAGACAAATACACACG
ACCATGGACCTCAGGGACCCACTGCAGGGAANGGCTTCCCTCCAGGAGAG
CTTCAGACCAAGAGACCCCAAGGGCTTGGGTAACCCACAGCAGTGGGGGC
AGTGCTCCACCACCCACCCTATGCATCCCTCCTCCCAGGTTGCCTGTCCC
AGGCAGGTTTGGCACCTGGAGCCCAAGGGTATCAAGTGTCTCTCAGACAC
AGAGCCGTCCCCCCACTGAGGCTCCCCCTCCTGCACAGGCGACAGGCTTT
GGGGGAGGGTCTTGGGCTTCTGTGGTTCAGGCAACTCTGTCCACTTCCCC
CTTTGTCCTGGCCACAGTGTCGTGGGAGCCAACACCCTTGCTGAAGTTCC
AGGAGCCCCTACCTGGAGAAGCCAGCCCCCTGGAGCTGGCGCTGTTGGTG
TTGTATCCAGGGCCCGGCCCAGAGGTCACTGTCACCGGGGCTGGGCTGCC
CGGCGCCCAGGTACCAGAGAGGTGAATGAGGCTGTCCCTGGGCCACCAGG
AGCCCTCATTCAAGGCAAGGGCGGGATTATTGAGGGGGGGGGNTAACTGC
ACCTAACAGAAAGGCTGTGACTGTCCAAGTTGGAATTTTGCAGGGATGTT
TAGGGCAGCAGGCAAGCAGGGCTGGTGTCCCAAGGCCCCAGCAAGCCTGG
CTGAGTCCCCATCTCCACAGAGCCTCTGCCCGACCAGGGACTCTGGCTTC
CTGGCGTTGGCGGTCGACCACCCAGAGAGGGCCTGGCGTGGCTCTGGGCT
TGCTCTGACCCTGCGACGCCGCGGAAATGGTAGCCCCCTCCCCCAGACTG
GAGCCGGGCTGGGGCGGCTGCCCTCGGAAACACCCCCCCCCCTTCCAGNC
GNTGAGCCAGCTCCTGCCTCCATCCTCAGGTGCCTCCCTGAGCACCGCCC
AGCTGCAGGCGCTGCTGTTTGGCGCCGACTCTCGCTGCTTCACACGGATG
ACCCCGGCCCTGCTCCTGTTGCCGCCGCAGGGGCCGGTGCCGATGCCCGC
ACACGGCCGGGTGGACTCAATGCCATTCCCGCAGCCCAGGCTGCGCATGA
GTCAGAACTTGGGGGCGCAGGGACGTGGGGGCAGCGCAGGCTTGTGCCCT
CACGTCCCCGCGCTCCGCCGTCCAGGCTGTCCCCAGAGCCC

TABLE 11

Coding and Non-coding Portions of Consensus Porcine
GPX4A (Exon 4- Intron 4-Exon 5-Intron 5- Exon 6)
(SEQ ID NO:27)

GAGCCAGGGAGTGATGCTGAGATCAAAGAATTTGCTGCTGGCTACAACGTC
AAATTTGATATGTTCAGCAAGATCTGTGTGAATGGGGACGATGCCCACCCTC
TGTGGAAGTGGATGAAAGTCCAGCCCAAGGGGAGGGGCATGCTGGGAAAG
TGAGTTGGGGGGCTGGGGTGAGAGTGGAGGGCAGTGGGGATCTGCAGCTGC
CACGGGATTACTGATGACACATTTCTTTTTGCAGTGCTATCAAATGGAACTT
TACCAAGGTAAGGGGGTGCTGAGGGCCCGGGGGGTGCCCTCAGTCACCCTG
GTGCCACTTCTAGGGTCTCCACCTGACCTAAATGGAGTGATGGGTGGGGGC
CGCTTGCTTGCTTGCCCCAGTCCCACCACGGTGGCCTTCTGTCCCTGACACC
ACCTGTCCTGCAGTTCCTCATTGATAAGAACGGCTGTGTGGTGAAGCGGTAC
GGTCCCATGGAAGAGCCCCAG

TABLE 12
Porcine FSHb gene sequence
(coding and non-coding regions)

| | |
|---|---|
| 5′ untranslated region | (SEQ ID NO: 28) |

GAATTCAGGA AAGAGGTCTT CTGTTCATTT AAAATATAAC GTGATGTGTG
TTAACACTGA GGTAGATACT GGGAATTAAG GAAACAATAG
AAAGTACTGG ACTGAGAATG AATACGGAAT ACTGTGTAAA
GTGGAACGAG TGAATGTCTC CTAGGGGAAG CTACATCTAA ATGGAATCTT
GTAGAAGTGT TTGTAGGAAT AGCTCAGATG AAAAGGAGAT
GAAAAAGGTA CCTCAGGCTT AAGGAATAGC CTGATTTTCA GAGGTGGGAA
GGTGCTTCAA GCCAATGAAG TGAGATTTTT TTTTTTTTTG GTCTTTTTAG
GGTTGCACCC ACAGCATATG GAAGTTTCCA AGCTAAGGTC GAATTGGAAC
TGCAACTGCC AACCTACGCC ACAGTCACAG CAACATGGGA TCTGAGCTGC
ATCTGTGAAC TACACTUCAG CTCATGGCAA CACCAGATCC TTAACCCACT
GAGCAAATCT AGAGATCAAA CCTGTGCCCT AATGGATACT AGCCAGGTTC
ACTACCACTG AGCCACAACG GTAACTACTG ACGTGAGAAT TTAACATAGG
ACCTCCTTAA ATAATGTTCA ACATTTTGTT TAAATATTGA GTTAATTAAT
ATTATTATAC TAGAACCCAG TAATAAAGGG CTAGAAATAA AAATGGGTAT
TATCAGTCAC CTTCTAACCA GGAAAACAGA AACTGCTCCT GATAAGAGAA
GTCAGAGGAT ATTTAATCTG GGGAATGCAT TACCTAAGTT TTAGAATTGT
TGAGAAGCCA GACAGGAAAT AAGGAAACCC AAAAATCAGT
AACCATGGGA AGCTCCCATC TACCCTCAGG ATTAGAGAGA
CACAAATGAG GTTCCTGGAG CCAAAAGGTG AGACCACCCA
GCAGAAGCTC AAGCCACATG TGGAGTTTCC TCACAAAAGC TGGGAACACT
GAGGGAGGAG CTGTCTGATG CAACCTGGAC CAAGGGAGAA
AGTGCAGCTA CTGACAAGGA AAGAATGTAA AGGAGAGACA
TACTCCAACC TTCTTCTTCT TTTCACTCTC TAATCTCCTT CCACAGAGAC
AAAAGGCTGC TGACACAGCA GCCTAAGAAA GGTAGCCTGC
AGAGGTCCCT TCTCCCAAAA ATCAGAGAGC AAAACAGGAC
AAGAACAAAA AATGTATCAG ATAGCAAACA GGCTATGGAC
AAGCACAACA GAAAGAAAAT CAGAGTGATC TATGTTTCAC TTAGTTCAAC
AAAAGTGTAT CAGTGCTGGA GTTCCCCTTG TGGCTCAGCA AAAACAAACC
TGACTAGTAT CCATGAGGAC TCACATTCCA TCCCTGGCCT CACTCAGTGG
GTTAAGGATC CAGCATTGCC ATGAGCTATG GTGTAGGCTG CAGACTCAGC
TCAGATCTGG TATTGCTGTG GCTATGGTGT AGGCCGGACG GTACAGCTCC
GATTCGACCC CACCTGAGAA TTTCCATATG CCACAAGTGC GGCCCTAAAA
AGACAAAAAA AAAAAAAAAA AAAAAAAAAA AAAAGAGTAT
TAGTGCCTAC TGTGGATTAG AAACCATTCC TATTGGGAAT ACAAAGGTGA
ATAAGAAAGC TCACTACATC TTCATCAATA AAATATTTTA ATAACTTTTG
TGAGAGCAGT AACATCTAAC TTGGAAATAC ACTTATCAGA TAAACTAGAC
TATAAGAGGC TTGACATTGT GAGAAAGGTC TGGGGCCTCG TGATAGGTCA
AGGAAAATAA GGTTATTTGG GGAAACCTCA GACCTAAATG
TGGATGGAAG TATAAATATG GACATTAGGA ATAGCTTCCC AAATTCTGGA
TGGCCTCTGT TTTGGCCCCT CTCCAACTAA TGCAGTTGGT GAGAATTATA
AACCACAGTA TGGTTCAATG AGTAGCTCTG TTTTGGAGAC CAGCAGACCT
AGATATGAAC CTTAGCCTTG CTCTTTCAGG TTCCATAGTT TTGGGCAAGT
CATTTAAATG TTTTCCCATC TTTCAAAGAG TAATAATAGT AACTCCTTTA
AAAAGTTGTT TAAAAATTAT ATGTGATCAT ATATTTGAAG TGTTTAAGTG
TCTGGGGCAT AGTAGGTGCT CAATAAAAAC CTGTTAATAT TTTAAATTGA
ATGTGAAAAG ATTGTATATA CATTACTCAT TAAAACACAT GAATTCAATA
TAGTCATATA AATATACTTT GTGAACACGC ATAGATAACA TAAAAAGAGT
TAATTTGAAA TATAAGGTGG GAATTCGTAC CATGGCACAG GGGGTTAATG
ATCCACTTGT CTCTGTGGCA TTGCTGGCTC AATTCCCAGC CTGGCTCAGT
GGGTAGGATC TGGCATTGCC ACAGCTATGG CATAGGCCAC AGATTCAACT
CGGATTCGAT CCCTAGCCTG GAAACTTCCA CATGCCACAG GTGCAGCCAT
TAAAAAAAAA AAAAAAAAAT TCTACATTCC TTATTACTTA CACAAGTGCT
AAATCAGCCC CCAGTACTTT GATAAGTTTT ATCTTTGTCA CACATGTTTG
ATAAAATCAT AACCCTGGAT AAATCCAAGT ATTTGTTACC CATGAGTCTG
AACTCCTGCC ATTAAATTAG GCAAAAAAAA AAAAAAAAAA
AAAATCATGT TTAGTGGTCT TGGGTTAAAT TTTTTTACCA TAAACCTCAA
ATGGTCCCTT AATACTGGTA GGCAATTTTA CTACCTATAC CTAACTCACC
AATGACTCAG TCCCTCTACC AGTCTCATAC AAATATTAAG CCTTGGATCT
CTCAATCCTC AACAATGCAT CCACTACCTT TACTCTCAGA TGATGATCTT
ACTTCCTACT TTACTGAGAA AATGAAAACA ATGACAGGAG AGTCTGTATA
AAGCCCATCA CCCACCAAAC ACTCACCATC TTCTGCACTC ACCACACCTC
CCCAACCAGC AGCATCTCTA CCCATGACTC TGCCTCCTGC CCACAACAGG
ATGAGCTCTC CTGTTTAAAG CCAGTCATTC TACTTGTGCT CTAAGATCCA
TCTCTTCTCA TAGTCTACCT AAGAACACTG AAGAAATTTT CCTCTCTTGC
TCCAACATCA TTTTTCTCTC AATCATTTGC ATCACCAAAC TAACAGTTAT
GTCTTCAGTC TTAAAACATA AAAATCAAAA GGAAATTATC TTTACCCCAC
TTCCATGTGA CCAAATCACC TGTTTTTTTC CTCATCTTTG TATCAAAATT
CTGGGGAGAA AAAGTTCAAC ACTTTTTTTG TAATGGTCAC ACCTGTGGCA
TATAGGAGCT CCTTTGCCAC AGCCACAGTA ATGCCGGACC CAAGTTGCAT
CTGCAACTGA CACGCAGTTT ATGGCAATGC GGATCCTTAG TCCACTGAGA
GAGGCCAGGG ATTGAATTTA TATCCTCAGG AAAACAATGC TGGGTTCTTA
ACTTGCTGAG CCAAATGTGA ACTCCTCAAT TCTTTTTTAT TCATTTCTTT
CCAATCACTC AGTCTGCTCT TTTATTGAAT TATAGCTGAT CTATAATGGT
ATGTTAGTTT CTGGTGTATA GCAAAGTGAT TCAGTTATAC ATACATATTA
CTTTTCACAT TCTTTTCCAT GACAGTTTAT CACAGGATAT TGAATATAGT
TGCGATACAG TAGGACCATT TTGTTTATCT ATCCTATATA TAATAGTGGT
TAATCCCAAA GTCCCAATCC AAACCATCCC CACCCTCCTG CCCTTGGCAA
CTACAAGTCT GTTCTCCATG TCTGTGAGTC TGTTTCTGTT CCATTCATTT
GTGTCATAAT TTAGATTCCA CATATAATTG TAATCATATG GTATTTGTCT
TTCTCTTTCT GACTTGCCTC ACTTAGTATG ACAATCCATG TAGCCACAAA
TGTCTTGACA ATTACTTAAA CACACACCAA TCAGGGTTTT GTTTCTCTCA
CTCCAAAGGA GCTTCTCTAG CCAAGGACAC TGGCAACATT TATGCTGCCA
CACGCATTGC TAACCTGTCA GCAGCATTTG GTACAGTTGT CACTTGCTCC
TCCTGACAAA CTGGCTTTAC TTGATTTCTG GGACACCACA TTCTCTCCAT
TCCTTTCTTT CCTCAATGAC CCTTCTGTTT CCTTTGGGCA AAGGAAGGGA
AAAAAACTTC ATCTTATTCT TGACCTCTTA ATATTAGCAC ACACCAGCCT
CCACTCTTGG TCCTTTTATC TTCTCTATTT ATACTTACTC CCTTGGTAAC
TTCTTCAAGG CTCATGCCAA TTATACATTT TAGCTAGCAT ATTTCTCCCA
AAATCCAGAT TCACCATTCT ACTTAGATAT CTTAAGCTCA ACCTATCCAT
ACCGAACTCC TTATCATTTT CCCAAACTTA CTATATTTAT AGCCATCCCA
TTTCAGTTGA TAACAAATTC ATCCTTCAAG TCACTCAGGC CAGAATCTTT
AGAGTCATCT TCACTCTTTT CTTTTTCTCA CACTCAGGAT TCATCCATCA
GAAAATCCTG CTGGCTCCAC TTTCAAAATA CATATGAAAT CAGATTACTT
TGATTATTTT ATTACTACTA TTACTGAACA GATAGCACTT CTCACCCAAG
TTGCTGCAAG AGCATCTAAT AGGACTTCCT GTTTCTACCT CCCCCACCCC CATATTAGCA
ACCAGGCAGC CAGAGGGTCC TTTAAGACTT AAACCTGATT ATATCACTCC
TATACTCAAA ACCCTGCAAC TGGTCCCCAA ACACCGACAG TAAAAACTGA
AGTCTTTACA TTGAACTAAA AAGTCCGACA TTATTTGACT TCTGCCACAT
CTGTGACATC ATATCCTCAT ATTTCCATCA TTGTTCCTTT TCTCCAGCCA
AGGAGCTTAA TTAATTAATA AGCTTAATTA ATTGCTCAAT TAATAAATAT
TTGTTAAATC AATCTCAGTT TCCATGGAGC TCATAGTCTA CTGGGAGAGA
AAAATATATA AAAGAATACA AAAAGAAGGT AATTAAAGCT TTCCTCAATC
TCCCATTCCT AAACAATGAC AAGTGAATGT TGAAGGTTGA GAAATTTGCC
AGGGGGTGGG AGTAGTATAG GGGACATTGG GAGGAAGCAA
GGACATTTCA GGAAGGATGA ACATGGCACA TACAAAGACC
TAGAGAAATG AATCAGCAGA ACATTTAAAG AATTACGAGT AAGCATCAA
AGAATAATAT TTAAGATTAA GGAATCTGAA TATGGGAAGT AAACATAAAT
ATAATTTACA CTTTATAAAA GAGTATAATC ATGAAAGACT CTCTATTTGT
TTCTTCCCTT ACAGCTGTCA GTCTAGTCTC AGAGTAACTT ATTAACCATA
TATATATATA TTTTTTGACA CACCTCAACA GTGCCAAAGC AATACTTGGA
AAGGATTCTA AATTCCCCAA ATTAAATATA CAAAAGAAAA ACCCAGAGTC
AGACTTAATT TGAAAAGGTA AAGGAGTGGG TGTTCTACTA TATCAAATTT
AATTTGTACA AAATCATCTC TGGTAACATT ATTTTTCCTG TTCCACTGTG
TTTAGACTAC TTTAGTAAGG CTTGATCTCC CTGTCTATCT AAACACTGAT
TCACTTACAG CCAGCTTCAG GCTAACATTG ATCTTACTAA TACCCAACAA
ATCCACAAAG TGTTAGTTTC ACATGATTTT GTATAAAAGG TGAACTGAGA
CTAGATTCAG CCC

| | |
|---|---|
| Exon 1 | (SEQ ID NO:29) |

ACAGCTTCCC CCAGACAAGG CAGCCGATCA CAG

| | |
|---|---|
| Intron 1 | (SEQ ID NO:30) |

GTGAGTCTTA GCATTTATAG TTACCAAGAG GTGACAGTTA GTTCTGAAAT
GNITTTTCGG GATCTGAAGA ACAAATCTAG AGCTTTTTAA CTTCTGTTGG
GGAGGGAATT CGTACTTGTC AACCTGGCTT CTCAAATATG GATAGTGCAC
TGTAATTACT GTAGCAAGCA ATTGACTTTT CATAGACCAG TTCACCTAGC
CTCTGATATG GTCTTATTTT ACAAAAAGGA GGAAAAAGCA AATGATATTT
ATGAGATGCT AAAAATGATG AACTAATTTA GTAGTACAAA AGTTTTTCTT
GGAGTTCCCA TCGTGGCGCA ATGGTTAACG AATCCGACTA GGAACCAAGA
GGTTGCGGGT TCGATCCCTG GCCTTGCTCA GTGGGTTAAG GATCCAGCAT TGCTGTGAGC TGTGGTGTAG
GTTACAGACA CAGCTTGGAT CCCACGTTGC TGTGGCCCTG GCATAGGGCG
ATGGCTACAG CTCTGATTAG ACCCCTAGCC TTGGAAACTC CATATGCCAA
GGGAGCAGTC CAAGAAATGG CAAAAAGACC AAAAAAAAAA
GTTTTTCTTT TTAAATAAAA TGTTTTAAAA TGATAATGAA GGGACAAATA
TGATGATCAC AATTACTTGC TTCAGAGTAA TCCTTTAAGA CAGTCAATGG
CAATACTCTA TAAATATTGC TCTGCTTAAA ACATTATATT GGAGTTTTGA
CCCATAATAT AGTTCTACTT TGACAAAAAA AAAAAAAATT
GAGGAGGAGA ATAAGAAGAA ACGTTT
GGAGTTCCCCGTCGTGGCGCAGTGGTTAAACGAATCCGATTAGGAACCATG
AGGTTGCGGGTTCGGTCCCTGCCCTTGCTCAGTGGGTTAATGATCCGGCGTT
GCATGAGCTGTGGTGTAGGTTGCAGACGAGGCTCGGATCCCCGCGTTGCTGT
GGTTTCTGGCGTAGGCGGGTGGCTACAGTTTTGATTCGACCCCTAGCCTGGG
AACCTCCATATGCCGCGGGGAGCGCCCAAAGAAATGGCAAAAGACAGAAA
AAAAAAAAAAAAAAAAAAAAAAAAAAAAGAAACGTTT¹
GTTCAAGAAA CAAGAATFFAA GAAAAGGAAA GGAAGGAAAA
CCACTATGGA GTAAAAGTGA CTGGAGAGGA TGAATAGACC AGTTATTCAA
GGTTTGGTCA ACTTACATTA CGAATGTAAT TCTTTGGTTT TTCAG

| | |
|---|---|
| Exon 2 | (SEQ ID NO:31) |

TTTTTTACAG GCCTTAATTG TTTGGTTTCC ACCCCAAGAT GAAGTCGCTG
CAGTTTTGCT TCCTATTCTG TTGCTGGAAA GCCATCTGCT GCAATAGCTG
TGAGCTGACC AACATCACCA TCACAGTGGA GAAAGAGGAG TGTAACTTCT
GCATAAGCAT CAACACCACG TGGTGTGCTG GCTATTGCTA CACCCGG

| | |
|---|---|
| Intron 2 | (SEQ ID NO:32) |

GTAGGTTCTT TGCTTTGCTA GAAGTGAGGG TGCTGAAGGT CTGTAAAAGG
CGGGCTTTAC TAATTCCCAC TTTATCAATA TTTTAAGTTT CCGGAACAGC
CATGAGTCCC TTAGTCAATA CTGTCTGTTT CCTGATTGGG GTTATTTACC
ATGACATCGG TTAAATCTTC AGGCCTGGAT TTGATTAAGG TAAATTTAGG
GAAGCCTCAG ATTTTATCTG ATTAATTTGG TAATTGCCAA CTCTATTTTT
TAATTTTATT TAATTTTTTT ATTTCAAAAA AAGTAGTTCT ATTCTAGATT
CTACACATAC AGAGATAAAC ACATAAACAT ACATATATTT AATAACAGAA
GATCTACAAT ATTTCCCAAA AGCCAATTTT TGTAATTGAA GCTATATCTT
TGCAATAGAG ATAGTATCAA AATGTTTGTA GCAACATAAA AACACAGCCA
TGTTATAAAA ACTGTCTTAC TGGCCCATCT CAATACAAAT GCCAACGCGC
AGCCTGAGAA CACAATCAAT CCYJGCAGAC TGTTAGGACC CAAATGAACT
GGCAAACCCA CTCCCTTCTT TATATGGTTG AGAAAAACAA GGCACAGAGG
GATAAAACCA CTAGTTTGTA TTCACACAGT TTCTTTGAAT TAATCCAAGT
GAAAAAGCAG TTTCTACTTT ATTTTTTCCC CTATAACACC TGGATATCGA
TGCAGAATTT CCGTAAATTG AAATTGAAAA CAACTTTTTA ATGCAATATA
CTTTACTGGG TGGTAAATGA GTTTGACCAA ACTCCACTTA TTGCATCTTA
TTGGGATACA GACTTGATGG CATGATATGG AAATAAATTA AACATAAGTG
TCTATTTCTT CCCTCAGTGG ATTTTTTTTT TTAACTAGAA AGTGTTAGAA
TAAGGTTGTT CTGACAGGAC TGAAGTTCTT ATACACAAAC ATGAAAGCTT
TGAAACTGAG CTCTGAAAAA TATACAGCAT TTAAGAGGGG AAGATGTCTG
TAAGACAGCA GAATATTTAA AATCTTACAT GAATTTTTAT AGTCATGTTA
AGCTAAGTAT TAACATTCCA CATTATATAT TTTTGATTTT TTTTATACAC
ACCCAGGGAC CATGTATTGA GAAAATTTTT CTGAGAAATT AAACTTCAGT
TTTTTATGGG TTAAGCTGTC ATTAATATAG CTTTCAACTT AGTAATTAAT
ATAGCTTTCA ACTTTCAAAA CGTCAAAATT TCTGTCCTAT TTTCTTTTTA
ATTATTTTTT ATATTGAAAG TTAAGTTTCT TTAAAGTCAG AGAAATAATT
AACATTTTGA CATAGACATA AGGAGTAGGA AAAGGAATAA TACATTTTCT
GTAAGATTTC CAGATCAGAA AACATGGCAT AGCATATAGG TTATTTATGA
TTTATGAAAT CATGTTTCCT TGGTTAGGAA TTCTATAAAT GGCCTTAATG
GATAAATGTC AGAGCAAGAA ATATTCAATG CCTGTCTCAT TTTGATTAAA
TAGAAACTTC TGTAATACTT TAACCTAACT CTCTCTCTCT CCCCTGAATC CCTTAG

| | |
|---|---|
| Exon 3 | (SEQ ID NO:33) |

GACCTGGTAT ACAAGGACCC AGCCAGGCCC AACATCCAGA
AAACATGTAC CTTCAAGGAG CTGGTGTACG AGACCGTGAA AGTACCTGGC
TGTGCTCACC ATGCAGACTC CCTGTATACG TATCCAGTAG CCACTGAATG
TCACTGTGGC AAGTGTGACA GTGACAGTAC TGACTGCACC GTGAGAGGCC
TGGGGCCCAG CTACTGCTCC TTCAGTGAAA TGAAAGAATA AAGAGCAGTG
GACATTTCAT GCTTCCTACC CTTGTCTGAA GGACCAAGAC GTCCAAGAAG
TTTGTGTGTA CATGTGCCCA GGCTGCAAAC CACTATGAGA GACCCCACTG
ATCCCTGCTG TCCTGTGGAG GAGGAGCTCC AGGAATGCAG AGTGCTAGGG
CCTCAGTCCC ATCACCACTC AACCCTGTATTTTGGGTCTG GTTCCATAAG
TTTTATTCGG TCTTTTTTTT TTAAATTACT CAATGAATTT TATTACATTT
ATAATTGTAC AATGATCATC ACAACCCAAT TTTATAGGAT TTCCATCCCA
AACCCCCAGC ATAGACCCCC ATCTCCCAAT CTGTCTCATT TGGAAACCAT
AAGTTTTTCA AAGTCCGTGA GTCAGTATCT ACTCAGTCTT ATTACCTTAA
TGACATGTGG GTGTTTTCTG TTTAATAATC TTAGAAATCC TCTCAAGACA
GGGATATGGA CCCAGAGGAA GGAAATGGGC TAAGAATGGG
TGAAAGGACT AAATGCAGCA TTCTCCCACT AGACACAGAA
GCCTACAAGA GCAGGGCCAG TCTCTTTGTC ATGAGTGTGG CC

| | |
|---|---|
| 3′ UTR | (SEQ ID NO:34) |

TCAATACCTA GCACAGTGAC TAGAATTCAG TAAGAAACTC AAGAATGGCT
TCCTTAAGGA AAGTAAGATT GGAAATGTAG GGGGTAGGAA
AATACTGAAA GAAGATGTTG GAGGCTATGT GATGAGGCTG CCCTTGGCAA
TGCCAGTCAG CCCGTGGAAG GGGGTCCATC AGTTCCAGTA CCGCTTCACC
GCTCTTCCTC CGGCATATGG AGGATGGAGA CAGGACATCT CTCTCAGGCA
GGTGGCGGTT ACCGAGCTCA GGATTTCCAA CCCCTTTAGT TAAGGGCAAA
AGCAAGAAAT GTTAATGCGG GTTTGTGGAA ATTAACCCAC ATCTATTCCA
TCATTTAAAT AAATGGAACA AATGCTATCA GACTCCTGCA AAACTCCCTC
CAGGTTGGGA TCCACTCCTT TGGAGAGAGG
TGGATTTGAA AGCAGGTTTA AAAGCGATTT TGGCAACTTA ATAAGTACAT
TTATCTTATC TAAAAATGCA TTTGTGTAAA GAAATAGCTC TTTTAGAATT
AGCCATAAGG GGAAAAAAAC AAACAAAAAA AACTGCTGTT
TTCTAGAATA CTCTATCAGT CTTTTGTCTA TCCATGTTCT CACAAATCTA
TTTCTTTCAA GAAGGTAAAT CTTGAAGCTA TTTCATGAGT TGATGTTGTT
TTAAGATGTT ACCTCTTAGT TATGTACTTG TTTCATACTT ATGTTGTTTA
ATTTATTTAA ATCTTATTTT TTTAATAAAG ACGCTAGCTA CTAGAGTCAT
AGATTTGGAT TTTTTTCATA TACCAGCAGA TGACTAAAAT GTCTGTATAT
TTATAATATT AATAGAAAGA GTCTTATTTA
AAAAAACTCC TTGGAGTTCC CGTCGTGGCG CAGTGGTTAA CGAATCCGAC
TAGGAACCAT GAGGTTGCGG GTTCGGTCCC TGCCCTTGCT CAGTGGGTTA
ACGGTCCGGC GTTGCCATGA GCTGTGGTGT AGGTTGCAGA CGCGGCTCGG
ATCC

¹The underlined area is the insertion/deletion polymorphism in FSHb

TABLE 13
Summary of FSHb SNP

| | |
|---|---|
| Type of | Association |

| | | | | |
|---|---|---|---|---|
| Gene | Region | SNP | Change of nucleotide | with hernia |

| | | | | |
|---|---|---|---|---|
| FSHb | Intron 1 | Insertion | GGAGTTCCCCGTCGTGGCGCAGTGGTTA | Yes |
| | | /deletion | AACGAAT | |
| | | | CCGATTAGGAACCATGAGGTTGCGGGTT | |
| | | | CGGTCCC | |
| | | | TGCCCTTGCTCAGTGGGTTAATGATCCG | |
| | | | GCGTTGC | |
| | | | ATGAGCTGTGGTGTAGGTTGCAGACGA | |
| | | | GGCTCGGA | |
| | | | TCCCCGCGTTGCTGTGGTTTCTGGCGTA | |
| | | | GGCGGGT | |
| | | | GGCTACAGTTTTGATTCGACCCCTAGCC | |
| | | | TGGGAAC | |
| | | | CTCCATATGCCGCGGGGAGCGCCCAAA | |
| | | | GAAATGGC | |
| | | | AAAAGACAGAAAAAAAAAAAAAAAAA | |
| | | | AAAAAAAAA | |
| | | | AAAAAGAAACGTTT (SEQ ID NO:35) | |

Table 14. FSHb PCR Test Protocols

| | | |
|---|---|---|
| Forward primer: | FSHbF 5′-CCT TTA AGA CAG TCA ATG GCA A -3′ | (SEQ ID NO:36) |
| Reverse primer: | FSHbR 5′-AGT GGT TTT TCC TTC CTT TTC C -3′ | (SEQ ID NO:37) |

PCR reagents:

| | |
|---|---|
| Extract-N-Amp PCR ready mix | 5.0 μl |
| FSHbF (5 μM) | 0.5 μl |
| FSHbR (5 μM) | 0.5 μl |
| QH₂O | 2.0 μl |
| Genomic DNA | 2.0 μl |
| | 10.0 μl |

PCR program using PE9700:

94° C. 30 Secs

95° C. 5 min 55° C. 30 Secs×40→72° C. 7 min 4° C. ∞

72° C. 45 Secs

(9600 Ramp)

This test is an insertion test so there is no digestion.

Load and run on 3% NuMe Agarose at 150 volts for 45 min

FIG. 9 shows the band sizes expected.

The Advantage of Combining the Two SNPs (MIS/HaeIII and FSHb)(One SNP Per QTL) on Hernia Incidences.

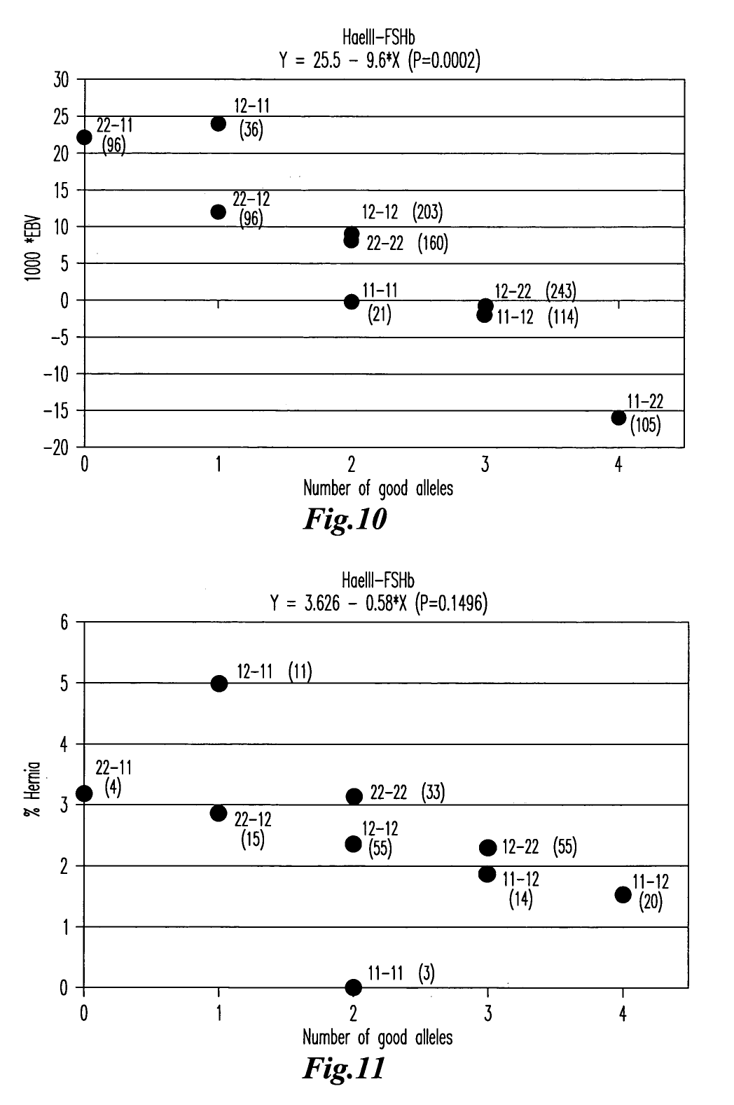

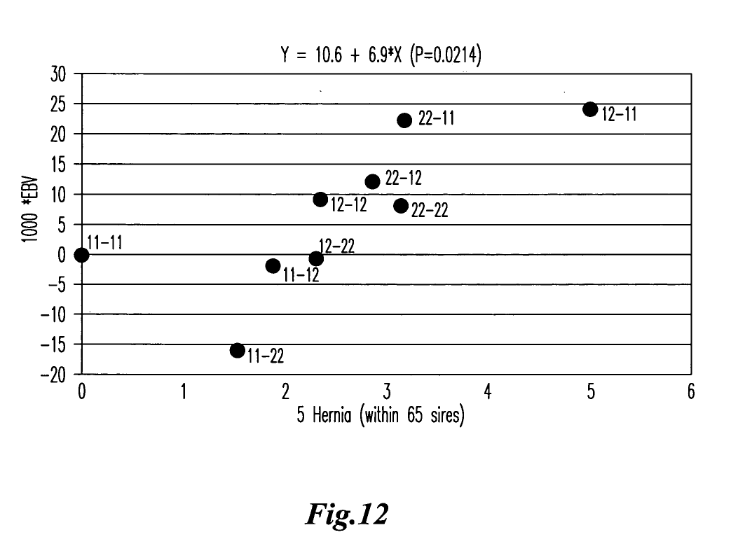

We used two datasets: the EBV dataset (1000 animals with estimated EBV) for hernia) and new sires (197 sires with information on hernia within their progeny). The two SNPs that were used are MIS/HaeIII (36 cM) and FSHb (7 cM). FIG. 10 shows that there is a clear linear relationship between the number of good alleles and hernia-EBV (R²=0.871). The 11-22 genotype is the favorable genotype, 22-11, 22-12 and 12-11 are the worst genotypes.

Referring to FIG. 11 (R²=0.272; when 11-11 excluded R²=0.505), if we ignore the 11-11 genotype, calculated based on only three (3) sires, the results are in agreement with FIG. 10. Namely 11-22 is the best genotype with the lowest hernia incidences. 22-11, 22-12 and 12-11 are the worst genotypes. Results from FIG. 10 are less accurate if the EBVs were based on previous generations (parents) rather than on current information from progeny as in FIG. 11.

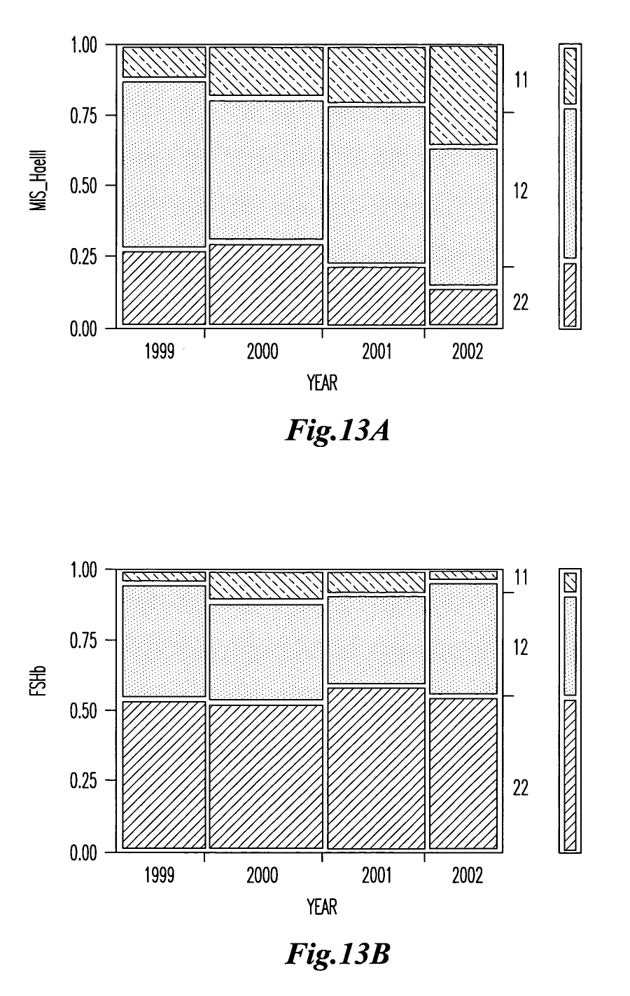

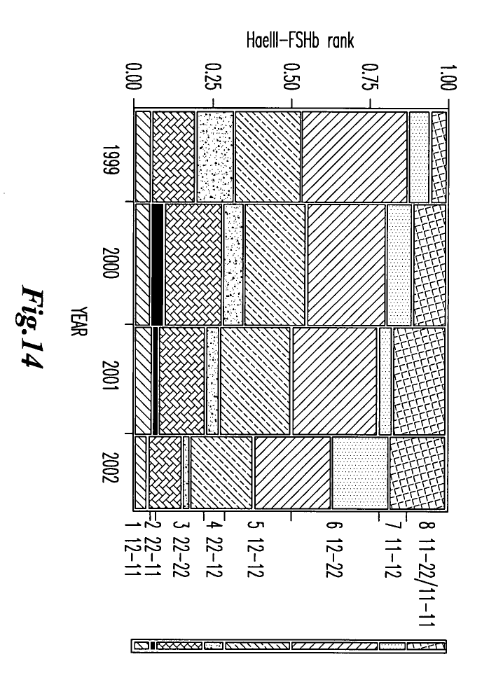

The inventors have thus established that there are two genomic regions on chromosome 2 are affecting hernia. Many markers have been developed within the two regions, the most promising results are from MIS/HaeIII and FSHb. A linear negative relationship between the number of good alleles of these two markers and hernia incidences has been established. The incidences of hernia were significantly lower in the good genotype combinations. As seen in FIGS. 13A, 13B and 14, with the successful EBV based selection against hernia, the number of the good genotype combination is steadily increasing.

What is claimed is:

1. A method for screening animals for scrotal hernias, the method comprising:
obtaining a sample of genetic material from an animal; and
screening for the presence in the sample of a genotype that is associated with scrotal hernias.

2. The method of claim 1, wherein the genotype is characterized by a polymorphism in the MIS gene or its equivalent as determined by a BLAST comparison.

3. The method of claim 2, wherein the polymorphism is located in an intron of the MIS gene or its equivalent as determined by a BLAST comparison.

4. The method of claim 2, wherein the polymorphism is located in an exon of the MIS gene or its equivalent as determined by a BLAST comparison.

5. The method of claim 3, wherein the intron is intron 1 or its equivalent as determined by a BLAST comparison.

6. The method of claim 3, wherein the intron is intron 3 or its equivalent as determined by a BLAST comparison.

7. The method of claim 5, wherein the polymorphism is a restriction fragment length polymorphism identified by the restriction enzyme HaeIII or one of its isoschizomers.

8. The method of claim 7, wherein the HaeIII-restriction fragment length polymorphism is the result of a substitution of adenine with guanine at nucleotide position 295 of intron 1 or its equivalent as determined by a BLAST comparison.

8. The method of claim 5, wherein the polymorphism is a restriction fragment length polymorphism identified by the restriction enzyme PmlI or one of its isoschizomers.

9. The method of claim 8, wherein the PmlI-restriction fragment length polymorphism is the result of a substitution of cytosine with thymine at nucleotide position 293 of intron 1 or its equivalent as determined by a BLAST comparison.

10. The method of claim 6, wherein the polymorphism is the result of an insertion of the sequence ACCAC between nucleotide positions 62 and 63 of intron 3 or its equivalent as determined by a BLAST comparison.

11. The method of claim 10, wherein the insertion is revealed as a restriction fragment length polymorphism identified by the restriction enzyme ScrFI or one of its isoschizomers.

12. The method of claim 4, wherein the exon is exon 3 or its equivalent as determined by a BLAST comparison.

13. The method of claim 12, wherein the polymorphism is a restriction fragment length polymorphism identified by the restriction enzyme BsaJI or one of its isoschizomers.

14. The method of claim 13, wherein the BsaJI-restriction fragment length polymorphism is the result of a substitution of cytosine with thymine at nucleotide position 15 of exon 3 or its equivalent as determined by a BLAST comparison.

15. The method of claim 1, wherein the genotype is characterized by a polymorphism in the GPX4A gene or its equivalent as determined by a BLAST comparison.

16. The method of claim 15, wherein the polymorphism is located in an intron of the GPX4A gene or its equivalent as determined by a BLAST comparison.

17. The method of claim 16, wherein the intron is intron 4 or its equivalent as determined by a BLAST comparison.

18. The method of claim 16, wherein the intron is intron 5 or its equivalent as determined by a BLAST comparison.

19. The method of claim 18, wherein the polymorphism is a restriction fragment length polymorphism identified by the restriction enzyme MseI or one of its isoschizomers.

20. The method of claim 19, wherein the MseI-restriction fragment length polymorphism is the result of a substitution of a guanine with an adenine at nucleotide position 68 of intron 4 or its equivalent as determined by a BLAST comparison.

21. The method of claim 20, wherein the polymorphism is a restriction fragment length polymorphism identified by the restriction enzyme AvaI or one of its isoschizomers.

22. The method of claim 21, wherein the AvaI-restriction fragment length polymorphism is the result of a substitution of a cytosine with a thymine at nucleotide position 21 of intron 5 or its equivalent as determined by a BLAST comparison.

23. The method of claim 1, wherein the genotype is characterized by a substitution of adenine with guanine at nucleotide position 295 in SEQ ID NO:3 or its equivalent as determined by a BLAST comparison.

24. The method of claim 1, wherein the genotype is characterized by a substitution of cytosine with thymine at nucleotide position 293 in SEQ ID NO:3 or its equivalent as determined by a BLAST comparison.

25. The method of claim 1, wherein the genotype is characterized by a substitution of cytosine with thymine at nucleotide position 15 in SEQ ID NO:6 or its equivalent as determined by a BLAST comparison.

26. The method of claim 1, wherein the genotype is characterized by an insertion of the sequence ACCAC between nucleotide positions 62 and 63 in SEQ ID NO: 7 or its equivalent as determined by a BLAST comparison.

27. The method of claim 1, wherein the genotype is characterized by a substitution of guanine with adenine at nucleotide position 68 in SEQ ID NO:19 or its equivalent as determined by a BLAST comparison.

28. The method of claim 1, wherein the genotype is characterized by a substitution of cytosine with thymine at nucleotide position 21 of SEQ ID NO:21 or its equivalent as determined by a BLAST comparison.

29. A method for screening animals for scrotal hernias, the method comprising:
obtaining a sample of genetic material from an animal; and
screening for the presence in the sample of a genotype that is associated with scrotal hernias, wherein the genotype is characterized by one or more of:
(i) a substitution of adenine with guanine at nucleotide position 295 in SEQ ID NO:3 or its equivalent as determined by a BLAST comparison;
(ii) a substitution of cytosine with thymine at nucleotide position 293 in SEQ ID NO:3 or its equivalent as determined by a BLAST comparison;
(iii) a substitution of cytosine with thymine at nucleotide position 15 in SEQ ID NO:6 or its equivalent as determined by a BLAST comparison;
(iv) an insertion of the sequence ACCAC between nucleotide positions 62 and 63 in SEQ ID NO: 7 or its equivalent as determined by a BLAST comparison;
(v) a substitution of guanine with adenine at nucleotide position 68 in SEQ ID NO:19 or its equivalent as determined by a BLAST comparison;
(vi) a substitution of cytosine with thymine at nucleotide position 21 of SEQ ID NO:21 or its equivalent as determined by a BLAST comparison; and
(vii) an insertion of a polynucleotide sequence as set forth SEQ ID NO:35 into SEQ ID NO:30 or its equivalent as determined by a BLAST comparison.

30. The method of claim 29, wherein the genotype is characterized by a substitution of adenine with guanine at nucleotide position 295 in SEQ ID NO:3 or its equivalent as determined by a BLAST comparison.

31. The method of claim 29, wherein the genotype is characterized by a substitution of cytosine with thymine at nucleotide position 293 in SEQ ID NO:3 or its equivalent as determined by a BLAST comparison.

32. The method of claim 29, wherein the genotype is characterized by a substitution of cytosine with thymine at nucleotide position 15 in SEQ ID NO:6 or its equivalent as determined by a BLAST comparison.

33. The method of claim 29, wherein the genotype is characterized by an insertion of the sequence ACCAC between nucleotide positions 62 and 63 in SEQ ID NO: 7 or its equivalent as determined by a BLAST comparison.

34. The method of claim 29, wherein the genotype is characterized by a substitution of guanine with adenine at nucleotide position 68 in SEQ ID NO:19 or its equivalent as determined by a BLAST comparison.

35. The method of claim 29, wherein the genotype is characterized by a substitution of cytosine with thymine at nucleotide position 21 of SEQ ID NO:21 or its equivalent as determined by a BLAST comparison.

36. The method of claim 1, wherein the animal is a pig.

37. The method of claim 29, wherein the animal is a pig.

38. The method of claim 1, wherein the screening step comprises:
PCR-amplifying the sample with a forward and reverse primer, wherein the forward primer is SEQ ID NO:10 and the reverse primer is SEQ ID NO:11;
subjecting the PCR-amplified sample to the restriction enzyme HaeIII or a HaeIII isoschizomer such that a HaeIII-restriction pattern of the sample is generated;
detecting the HaeIII-restriction pattern; and
comparing the detected HaeIII-restriction pattern with a second HaeIII-restriction pattern obtained by using the primers of SEQ ID NO:10 and SEQ ID NO:11, wherein the second HaeIII-restriction pattern is associated with scrotal hernias.

39. The method of claim 1, wherein the screening step comprises:
PCR-amplifying the sample with a forward and reverse primer, wherein the forward primer is SEQ ID NO:12 and the reverse primer is SEQ ID NO:13;
subjecting the PCR-amplified sample to the restriction enzyme PmlI or a PmlI isoschizomer such that a PmlI-restriction pattern of the sample is generated;
detecting the PmlI-restriction pattern; and
comparing the detected PmlI-restriction pattern with a second PmlI-restriction pattern obtained by using the primers of SEQ ID NO:12 and SEQ ID NO:13, wherein the second PmlI-restriction pattern is associated with scrotal hernias.

40. The method of claim 1, wherein the screening step comprises:
PCR-amplifying the sample with a forward and reverse primer, wherein the forward primer is SEQ ID NO:14 and the reverse primer is SEQ ID NO:15;
subjecting the PCR-amplified sample to the restriction enzyme BsaJI or a BsaJI isoschizomer such that a BsaJI-restriction pattern of the sample is generated;
detecting the BsaJI-restriction pattern; and
comparing the detected BsaJI-restriction pattern with a second BsaJI-restriction pattern obtained by using the primers of SEQ ID NO:14 and SEQ ID NO:15, wherein the second BsaJI-restriction pattern is associated with scrotal hernias.

41. The method of claim 1, wherein the screening step comprises:
PCR-amplifying the sample with a forward and reverse primer, wherein the forward primer is SEQ ID NO:16 and the reverse primer is SEQ ID NO:17;
subjecting the PCR-amplified sample to the restriction enzyme ScrFI or a ScrFI isoschizomer such that a ScrFI-restriction pattern of the sample is generated;
detecting the ScrFI-restriction pattern; and
comparing the detected ScrFI-restriction pattern with a second ScrFI-restriction pattern obtained by using the primers of SEQ ID NO:16 and SEQ ID NO:17, wherein the second ScrFI-restriction pattern is associated with scrotal hernias.

42. The method of claim 1, wherein the screening step comprises:
PCR-amplifying the sample with a forward and reverse primer, wherein the forward primer is SEQ ID NO:23 and the reverse primer is SEQ ID NO:24;
subjecting the PCR-amplified sample to the restriction enzyme MseI or a MseI isoschizomer such that a MseI-restriction pattern of the sample is generated;
detecting the MseI-restriction pattern; and
comparing the detected MseI-restriction pattern with a second MseI-restriction pattern obtained by using the primers of SEQ ID NO:23 and SEQ ID NO:24, wherein the second MseI-restriction pattern is associated with scrotal hernias.

43. The method of claim 1, wherein the screening step comprises:
PCR-amplifying the sample with a forward and reverse primer, wherein the forward primer is SEQ ID NO:23 and the reverse primer is SEQ ID NO:24;
subjecting the PCR-amplified sample to the restriction enzyme AvaI or an AvaI isoschizomer such that an AvaI-restriction pattern of the sample is generated;
detecting the AvaI-restriction pattern; and
comparing the detected AvaI-restriction pattern with a second AvaI-restriction pattern obtained by using the primers of SEQ ID NO:23 and SEQ ID NO:24, wherein the second AvaI-restriction pattern is associated with scrotal hernias.

44. The method of claim 38, wherein the animal is a pig.

45. The method of claim 39, wherein the animal is a pig.

46. The method of claim 40, wherein the animal is a pig.

47. The method of claim 41, wherein the animal is a pig.

48. The method of claim 42, wherein the animal is a pig.

49. The method of claim 43, wherein the animal is a pig.

50. An isolated polynucleotide comprising SEQ ID NO:25, wherein the cytosine at nucleotide position 618 is replaced with thymine.

51. An isolated polynucleotide comprising at least 20 contiguous nucleotides of SEQ ID NO:25, wherein the polynucleotide includes the nucleotide at nucleotide position 618, and wherein the cytosine at nucleotide position 618 is replaced with thymine.

52. An isolated polynucleotide comprising SEQ ID NO:26, wherein the cytosine at nucleotide position 1099 is replaced with thymine.

53. An isolated polynucleotide comprising SEQ ID NO:26, wherein the adenine at nucleotide position 1101 is replaced with guanine.

54. An isolated polynucleotide comprising SEQ ID NO:26, wherein the sequence ACCAC is inserted between nucleotide positions 1940 and 1941.

55. An isolated polynucleotide comprising at least 20 contiguous nucleotides of SEQ ID NO:26, wherein the polynucleotide includes the nucleotide at nucleotide position 1099, wherein the cytosine at nucleotide position 1099 is replaced with thymine.

56. An isolated polynucleotide comprising at least 20 contiguous nucleotides of SEQ ID NO:26, wherein the polynucleotide includes the nucleotide at nucleotide position 1101, and wherein the adenine at nucleotide position 1101 is replaced with guanine.

57. An isolated polynucleotide comprising at least 20 contiguous nucleotides of SEQ ID NO:26, wherein the polynucleotide includes the nucleotides at nucleotide positions 1940 and 1941, and wherein the sequence ACCAC is inserted between nucleotide positions 1940 and 1941.

58. An isolated polynucleotide comprising SEQ ID NO:27, wherein the guanine at nucleotide position 220 is replaced with adenine.

59. An isolated polynucleotide comprising SEQ ID NO:27, wherein the cytosine at nucleotide position 284 is replaced with thymine.

60. An isolated polynucleotide comprising at least 20 contiguous nucleotides of SEQ ID NO:27, wherein the polynucleotide includes the nucleotide at position 220, and wherein the guanine at nucleotide position 220 is replaced with adenine.

61. An isolated polynucleotide comprising at least 20 contiguous nucleotides of SEQ ID NO:27, wherein the polynucleotide includes the nucleotide at position 284, and wherein the cytosine at nucleotide position 284 is replaced with thymine.

62. The method of claim 1, wherein the genotype is characterized by a polymorphism in a genomic region that is linked to a gene selected from the group consisting of MIS and GPX4A.

63. The method of claim 62, wherein the genomic region is a microsatellite marker selected from the group consisting of SW240, SW1686, SW1564, SW747, S0091, SWR1342, SW776, and SO_{226.}

64. The method of claim 62, wherein the genomic region is a gene selected from the group consisting of CGRP, FSHb, INSL3, PDE4A, RSTN, and CAST.

65. The method of claim 1, wherein the genotype is characterized by a polymorphism in the FSHb gene or its equivalent as determined by a BLAST comparison.

66. The method of claim 65, wherein the polymorphism is located in an intron of the FSHb gene or its equivalent as determined by a BLAST comparison.

67. The method of claim 66, wherein the intron is intron 1 or its equivalent as determined by a BLAST comparison.

68. The method of claim 67, wherein the polymorphism is the result of an insertion of a polynucleotide sequence as set forth SEQ ID NO:35 into intron 1 or its equivalent as determined by a BLAST comparison.

69. The method of claim 1, wherein the genotype is characterized by an insertion of a polynucleotide sequence as set forth SEQ ID NO:35 into SEQ ID NO:30 or its equivalent as determined by a BLAST comparison.

70. The method of claim 29, wherein the genotype is characterized by an insertion of a polynucleotide sequence as set forth SEQ ID NO:35 into SEQ ID NO:30 or its equivalent as determined by a BLAST comparison.

71. The method of claim 1, wherein the screening step comprises:
PCR-amplifying the sample with a forward and a reverse primer, wherein the forward primer is SEQ ID NO:36 and the reverse primer is SEQ ID NO:37;
detecting the PCR-amplified sample; and
comparing the detected pattern with a second pattern obtained by using the primer of SEQ ID NO:36 and SEQ ID NO:37, wherein the second pattern is associated with scrotal hernias.

72. The method of claim 71, wherein the animal is a pig.
